(19) United States
(12) Patent Application Publication
(10) Pub. No.: US 2024/0261218 A1
(43) Pub. Date: Aug. 8, 2024

(54) BIODEGRADABLE MUCOADHESIVE PHARMACEUTICAL FORMULATIONS AND METHODS THEREOF

(71) Applicant: THE TEXAS A&M UNIVERSITY SYSTEM, College Station, TX (US)

(72) Inventors: Robert Y. TSAI, College Station, TX (US); Xiaohua LIU, College Station, TX (US)

(21) Appl. No.: 18/562,374

(22) PCT Filed: May 13, 2022
(86) PCT No.: PCT/US2022/029145

§ 371 Date: Nov. 20, 2023

Related U.S. Application Data
(60) Provisional application No. 63/193,229, filed on May 26, 2021.

(51) Int. Cl. A61K 9/00; A61K 9/70; A61K 31/282; A61K 31/365; A61K 31/5377; A61K 47/32; A61K 47/38; A61K 47/42

(52) U.S. Cl. CPC A61K 9/006; A61K 9/7007; A61K 31/282; A61K 31/365; A61K 31/5377; A61K 47/32; A61K 47/38; A61K 47/42

(57) ABSTRACT

The present disclosure provides pharmaceutical formulations comprising i) a support frame, ii) one or more bioadhesive materials, iii) a first active ingredient, and iv) a second active ingredient. The disclosure also provides methods of treating a subject using the pharmaceutical formulations for various disease states and processes for making the pharmaceutical formulation.

### CROSS-REFERENCE TO RELATED APPLICATIONS

This application claims the benefit under 35 USC § 119(e) of U.S. Provisional Application Ser. No. 63/193,229, filed on May 26, 2021, the entire disclosure of which is incorporated herein by reference.

### BACKGROUND AND SUMMARY OF THE INVENTION

Oral cancer (OC) is a common cancer type in the United States. The five-year survival rate of OC is only 66%, representing one of the lowest rates among all major cancer types. Even after undergoing successful treatment, surviving OC patients often have to live with impaired functions and disfigurations that are caused by the treatment.

More than 90% of OCs are oral squamous cell carcinomas (OSCCs) that may develop from clinically visible leukoplakia or erythroplakia. Leukoplakia is widespread, affecting 1.5-4.3% of the world population. The malignant transformation rates of oral premalignant lesions (OPLs) correlate with the grade of oral epithelial dysplasias (OED), occurring at 4-11% for low-grade OEDs and 20-43% for high-grade OEDs. While some low-grade OEDs may spontaneously regress, high-grade OEDs typically persist.

Given the high mortality rate and treatment-related complications for OC, the most cost-efficient way to manage OCs is to eradicate OEDs before they become cancerous. Current therapeutic options for OEDs include surgical resection, laser ablation, cryotherapy, and administration of systemic medications. However, OC recurrence rates remain substantial—approximately 9-35% for surgical or laser treatment and 47-56% for non-surgical intervention. Furthermore, surgical excision and laser ablation of large-sized lesions require specialized clinics and can also cause severe discomfort and scar formation in many patients. Moreover, treatment with systemic medications is limited by systemic side effects and high recurrence. Accordingly, there is a critical unmet need for an effective non-invasive treatment for treatment of OC and OEDs.

Accordingly, the present disclosure provides pharmaceutical formulations that can be utilized for improved delivery to patients, including oral administration. Furthermore, the present disclosure provides methods of administering pharmaceutical formulations to subjects to treat disease states as well as methods of preparing the pharmaceutical formulations to achieve therapeutically beneficial results. Processes for making the pharmaceutical formulations are also provided.

The formulations and methods of the present disclosure provide several benefits to patients. First, the formulations comprise two active ingredients (oxaliplatin [OXP] and mycophenolic acid [MPA]/mycophenolate [MPS]) that demonstrate a synergistic effect in killing oral dysplastic keratinocytes and oral squamous cell carcinoma cells. Second, the formulations are designed for oral administration to patients to beneficially provide a higher tissue-to-plasma drug concentrations, a bypass of the hepatic first-pass metabolism, and a minimization of systemic toxicities.

Third, the formulations are created using a computer-aided design (CAD) three-dimensional (3-D) printing technique. Compared to conventional techniques (e.g., the solution casting/molding method), the 3-D printing technology described by the present disclosure is capable of fabricating the formulations to comprise a well-defined multilayer structure. Furthermore, the active ingredient composition in each layer of the formulation can be controlled with high level of precision. In turn, the formulations are able to achieve (1) a unidirectional release of active ingredients from the formulation to the oral mucosa of subjects and thus minimize damage to the surrounding healthy oral tissue; (2) a controlled and synchronized release of multiple active ingredients (i.e., oxaliplatin and mycophenolic acid/mycophenolate), and (3) a batch-to-batch consistency in the thickness of layers and the evenness of active ingredients within the formulation.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A shows a CELLINK_BIO_X_3 D printer used for fabrication of the exemplary pharmaceutical formulations. FIG. 1B shows the 3D printer printing an exemplary pharmaceutical formulation. FIG. 1C shows an exemplary double-layered pharmaceutical formulation comprising a top-to-bottom active ingredient ratio of 1-to-2. FIG. 1D shows an exemplary honeycomb pattern of a pharmaceutical formulation.

FIGS. 2A-2B show the in vitro release profiles for the first-generation (v1) formulations. The release profiles of oxaliplatin (OXP) (FIG. 2A) and mycophenolate (MPS) (FIG. 2B) from the pharmaceutical formulations are shown. Curves show the amount of active ingredient released from the formulation at specific time points as a percentage of the theoretical loaded amounts for OXP and MPS, respectively. Error bars show standard error mean (s.e.m). Sample size=3.

FIGS. 3A-3B show the in vitro release profiles for the second-generation (v2) formulations. The release profiles of OXP (FIG. 3A) and MPS (FIG. 3B) from the pharmaceutical formulations are shown. Curves show the amount of active ingredient released from the formulation at specific time points as a percentage of the total extractable amounts, which account for 66% and 9.4% of the loaded amounts for MPS and OXP, respectively. Error bars show s.e.m.

FIGS. 4A-4B show the in vitro release profiles for the third-generation (v3) formulations. Three types of pharmaceutical formulations were fabricated containing different doses of OXP: Formulation #1 contained 1.56 μg OXP and 10 μg MPS; Formulation #2 contained 5.2 μg OXP and 10 μg MPS; Formulation #3 contained 15.6 μg OXP and 10 μg MPS. The release profiles of OXP (FIG. 4A) and MPS (FIG. 4B) were quantified by LC-MS/MS and calculated as percentages of their respective total loaded amounts.

FIGS. 5A-5B show the in vitro release profiles for the fourth-generation (v4) formulations. Four versions of the v4 formulations designed with two different layers comprising bioadhesives/active ingredients. All four formulations contain the same total amount of OXP (7.1 μg) distributed in different ratios between the top and bottom layer, i.e., v4.1 (1:1), v4.2 (1:2), v4.3 (1:3), and v4.4 (1:4), and the same total amount of MPS (14 μg) distributed evenly in both layers. The in vitro release profiles of OXP (FIG. 5A) and MPS (FIG. 5B) from 0 to 4 hours were quantified by UPLC-PDA and calculated as percentages of their respective total extractable amounts.

FIGS. 6A-6B show the in vitro release profiles for the fifth-generation (v5) formulations. Four versions of the v5 formulations were designed with two different layers comprising bioadhesives/active ingredients. All four formulations contain two different amounts of MPS distributed in two different ratios between the top and bottom layers, i.e., v5.1 (14 μg in 1:1), v5.2 (21 μg in 1:1), v.5.3 (14 μg in 1:2), and v5.4 (21 μg in 1:2), and the same total amount of OXP (7.1 μg) distributed in a 1:2 ratio between the top and bottom layers. The in vitro release profiles of OXP (FIG. 6A) and MPS (FIG. 6B) from 0 to 4 hours were quantified by UPLC-PDA and calculated as percentages of their respective total extractable amounts. Error bars show s.e.m. (n=5).

FIGS. 7A-7B show exemplary pharmaceutical formulations comprising a two-layer design placed on a support frame.

FIGS. 8A1, 8A2, and 8B show the in vitro release profiles for the sixth-generation (v6) formulations. FIGS. 8A1 and 8A2 show variations of the v6 formulations designed to contain two layers of bioadhesives/active ingredients with the same total amount of MPS and different amounts of OXP. The cumulative release profiles of OXP (FIG. 8A1) and MPS (FIG. 8A2) into water solution from 0 to 4 hours (4 h) are presented percentages of their respective total extractable amounts. FIG. 8B shows ratios between the cumulative released amounts of MPS and OXP (in weight) by the v6 formulations over a 4-hour period. An MPS/OXP weight ratio of 6.9 (dashed line) equals an IC₅₀ ratio of 1.

FIGS. 9A-9C show the in vitro release profiles for the v6.4 formulation (normal dose) and the seventh-generation formulation (high dose). The v6.4 formulation contained a total amount of 1.059 μg OXP and 14.13 μg MPS in a 5 mm-diameter circular disc. The seventh-generation formulation contained a total amount of 1.59 μg OXP and 21.2 μg MPS in a 5 mm-diameter circular disc, representing a 50% increase over that of the v6.4 formulation. For both formulations, OXP and MPS were distributed in a 1:2 ratio between the top and bottom layer. The cumulative release profiles of OXP (FIG. 9A) and MPS (FIG. 9B) from 0-4 hours were quantified and calculated as percentages of their respective total extractable amounts. FIG. 9C shows ratios between the cumulative release amounts of MPS and OXP over a 4-hour period. An MPS/OXP weight ratio of 6.9 (dashed line) equals an IC₅₀ ratio of 1.

FIG. 10 shows the placement of the pharmaceutical formulations at various locations in the oral cavity of rats.

FIGS. 11A1, 11A2, 11B1, 11B2, and 11C show the in vivo release profiles of OXP and MPS for the OXP-MPS.v6.4 formulation. Rats were given 4 mm-diameter circular discs of the OXP-MPS v6.4 formulation on their dorsal tongue surfaces and then sacrificed after 0.5 h, 1 h, 2 h, 3 h, or 4 h (8 rats per group). Two additional groups were given a 4-hour application of the formulation sacrificed at 4 hours or 8 hours after the removal of the formulation. Samples were collected from tongues tissue. FIG. 11A1 shows the amount of mucosal MPS in the tongue over time and FIG. 11A2 shows the MPS release percentage over time. FIG. 11B1 shows the amount of mucosal OXP in the tongue over time and FIG. 11B2 shows the OXP release percentage over time. FIG. 11C shows the amount of MPS in plasma for measurement by LC-MS/MS (n=8). Arrows indicate the treatment time via the pharmaceutical formulation. Bars show mean±s.e.m.

FIG. 12 shows show a schematic diagram of the formulation treatment design using a 4NQO-treated rat model, including the time course of 4NQO treatment and incisional biopsy. The solid line at the top of the figure indicates continuous 4NQO treatment. The dashed line at the top of the figure indicates continuous 4NQO treatment if low-grade OEDs were not found. Thick arrows indicate two groups of rats receiving incisional biopsy in alternate weeks.

FIG. 13 shows drug interaction between MPA and OXP, 5-FU, or paclitaxel was determined by their respective combination indices (CIs). Five different drug-to-MPA ratios with different color labels were examined, with the 1-to-1 ratio representing each drug at equivalent amounts of their respective IC₅₀ concentrations. Numbers on top list the smallest CIs for each combination.

FIGS. 14A-14D show side leakage, mucosal permeation, mechanical strength, and drug stability of the OXP-MPS v6.4 formulations. FIG. 14A shows the cumulative leakage of OXP and MPS from the side of a 9 mm-diameter disc of the v6.4 formulation was measured at 10 minutes (10 m), 30 m, 1 hour (h), 2 h, and 4 h, and expressed as percentages of their respective total extractable amounts in the formulation. FIG. 14B shows the amounts of OXP and MPS passing through a porcine mucosal layer from an attached v6.4 formulation 15 (9 mm-diameter disc) measured at 10 m, 30 m, 1 h, 2 h, and 4 h and expressed as percentages of their respective total extractable amounts in the formulation. FIG. 14C shows the mechanical strength of the v6.4 formulation determined by its stress (σ, in MPa)-strain (ε) curve measured using a TestResources System. The line represents the best-fitting linear regression line of the initial slope of the stress-strain curve. Arrow indicates membrane breakpoint. FIGS. 14D1 and 14D2 show the amounts of OXP and MPS, respectively, remaining in the OXP-MPS formulation kept at 25° C., 4° C., or −20° C. for 1 day (1 d), 4 d, 7 d, 14 d, 21 d, 28 d, 60 d, 120 d, 180 d, 270 d, and 360 d, quantified and expressed as percentages of their respective amounts extracted from freshly made formulation s (n=5). OXP and MPS were measured by LC-MS/MS in (FIGS. 14A/B) or UPLC-PDA (FIG. 14D).

FIGS. 15A-15B shows that OXP is unstable in chloride-containing solutions. FIG. 15A shows stability of OXP dissolved in the NaCl (0.9%, 3%), KCl (3%), sodium phosphate (NaPi, 0.2M), glucose (Glu, 5%), and H2O solutions. FIG. 15B shows stability of OXP in solutions containing one of the formulation components, i.e., polyacrylic acid (PAA), DOPA, or CMC.

FIG. 16 shows the mechanical strength of four additional OXP-MPS v6.4 formulations determined by their stress (σ, in MPa)-strain (ε) curves measured using a TestResources System. Lines represent the best-fitting linear regression lines of the initial slopes of the stress-strain curves. Arrows indicate membrane breakpoints.

FIG. 17 shows the bioadhesive strengths of four OXP-MPS formulations on porcine mucosa ex vivo, measured using the TestResources System. The force required to detach the formulation from the mucosa was measured in newton (N) (Y-axis) and plotted against the probe position (X-axis, mm).

FIG. 18A shows the ex vivo residence time of the OXP-MPS v6.4 formulation (corners indicated by arrows) on top of a porcine oral mucosa. FIG. 18B shows the percentage of formulations adhering to the mucosal surface after immersion in simulated saliva for 1-to-4 hours (n=12).

FIG. 19 shows a schematic diagram depicting mucosal lesions (circle), incisional biopsy (triangle), formulation (“patch”; circle), and punch biopsy (dashed circle).

FIG. 20 shows a schematic diagram depicting the time course of formulation (“patch”) treatment (arrows), blood sample collection (arrow), and final punch biopsy (arrow).

FIGS. 21A-21B shows efficacy of the OXP-MPS v6.4 formulation in treating 4NQO-induced low-grade oral epithelial dysplasias (OEDs). Low-grade OEDs on the dorsal surface of the tongue were confirmed by incisional biopsies and treated with the OXP-MPS formulation (FIG. 21A) or the control blank formulation (FIG. 21B) for 2 hours×5 days. Dark arrows show dyskeratotic cells; lighter arrows show multi-nucleated cells.

FIGS. 22A and 22B show effects of the OXP-MPS formulation (n=9) vs. the control blank formulation (n=10) on the epithelial mitotic (Ki67) activity in the basal and parabasal layer analyzed by two-sided student t-test. Bar graphs show mean (±s.e.m); *, p<0.05; n.s., not significant.

FIGS. 23A-23D show the local and systemic toxicities of the OXP-MPS v6.4 formulation. Toxicities were evaluated 48 hours after the completion of the formulation treatment (4 hours×5 days) in healthy Sprague Dawley (SD) rats. FIG. 21A1-A4: Local toxicities were determined by H&E (FIG. 21A1), Ki67 (FIG. 21A2), and cleaved caspase 3 (CC3) (FIG. 21A3) staining of oral mucosa. FIG. 21A4 shows quantification of Ki67+ and CC3+ cells in tongue epithelium. Systemic toxicities were determined by liver function test (FIG. 21B) and complete blood count (FIG. 21C). Quantification of Ki67+ and CC3+ cells in intestinal epithelium (FIG. 21D). Scale bars show 100 μm in (FIG. 21A1) and 50 μm in (FIG. 21A2, FIG. 21A3). ALP, alkaline phosphatase; ALT, alanine aminotransferase; AST, aspartate aminotransferase; WBC, white blood cells; RBC, red blood cells. Bar graphs show mean (±s.e.m). Differences between the OXP-MPS (O-M) formulation (n=12) and the control blank formulation (n=10) group were analyzed by two-sided student t-test and determined as significant with p values≤0.05 (*) or not significant (n.s.) with p values>0.05.

FIGS. 24A-24C show evaluation of systemic toxicities 48 hours after the completion of treatment (4 hours of treatment with formulation per day for 5 consecutive days) by H&E, Ki67, and cleaved caspase 3 (CC3) staining of the intestine (FIG. 24A), kidney (FIG. 24B), and liver (FIG. 24C). Scale bars show 100 μm.

### DETAILED DESCRIPTION

Various embodiments of the present disclosure are described herein as follows. In an illustrative aspect, a pharmaceutical formulation is provided. The pharmaceutical formulation comprises i) a support frame, ii) one or more bioadhesive materials, iii) a first active ingredient, and iv) a second active ingredient.

In an embodiment, the pharmaceutical formulation is configured as a three-dimensional printed composition. In an embodiment, the support frame comprises plastic.

In an embodiment, the support frame comprises one or more polymers. In an embodiment, the polymer is polyurethane. In an embodiment, the polymer is polyethylene. In an embodiment, the polymer is polyester.

In an embodiment, the support frame comprises a polymeric film. In an embodiment, the polymeric film is a polyurethane film. In an embodiment, the polymeric film is a polyethylene film. In an embodiment, the polymeric film is a polyester film. In an embodiment, the support frame comprises a semi-permeable film.

In an embodiment, the one or more bioadhesive materials are selected from the group consisting of polyacrylic acid (PAA), gelatin-modified dopamine (DOPA), carboxymethyl cellulose (CMC), polylactic acid, sodium carboxymethyl cellulose, carbopol, chitosan, PEG (Polyethylene glycol), sodium alginate, gelatin, pectin, Poly(vinyl alcohol), Poly(ethylene oxide), Poly(vinyl pyrrolidone), methylcellulose, methylethyl cellulose, gum tragacanth, soluble starch, and any combination thereof. In an embodiment, the one or more bioadhesive materials are selected from the group consisting of polyacrylic acid (PAA), gelatin-modified dopamine (DOPA), carboxymethyl cellulose (CMC), and any combination thereof.

In an embodiment, the one or more bioadhesive materials comprises polyacrylic acid (PAA). In an embodiment, the one or more bioadhesive materials comprises gelatin-modified dopamine (DOPA). In an embodiment, the one or more bioadhesive materials comprises carboxymethyl cellulose (CMC). In an embodiment, the one or more bioadhesive materials comprises polylactic acid. In an embodiment, the one or more bioadhesive materials comprises sodium carboxymethyl cellulose. In an embodiment, the one or more bioadhesive materials comprises carbopol. In an embodiment, the one or more bioadhesive materials comprises chitosan. In an embodiment, the one or more bioadhesive materials comprises PEG (Polyethylene glycol). In an embodiment, the one or more bioadhesive materials comprises sodium alginate. In an embodiment, the one or more bioadhesive materials comprises gelatin. In an embodiment, the one or more bioadhesive materials comprises pectin. In an embodiment, the one or more bioadhesive materials comprises Poly(vinyl alcohol). In an embodiment, the one or more bioadhesive materials comprises Poly(ethylene oxide). In an embodiment, the one or more bioadhesive materials comprises Poly(vinyl pyrrolidone). In an embodiment, the one or more bioadhesive materials comprises methylcellulose. In an embodiment, the one or more bioadhesive materials comprises methylethyl cellulose. In an embodiment, the one or more bioadhesive materials comprises gum tragacanth. In an embodiment, the one or more bioadhesive materials comprises soluble starch.

Oxaliplatin, also known as [SP-4-2-(1R-trans)]-(1,2-cyclohexanediamine-N,N′)[ethanedioato(2-)-O,O′]platinum, is a compound that is an antineoplastic agent. The chemical structure of oxaliplatin is:

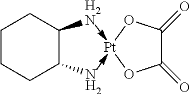

As used herein, the term “oxaliplatin” refers to oxaliplatin base, pharmaceutically acceptable salts of oxaliplatin, other salts of oxaliplatin, metabolites of oxaliplatin, and prodrugs of oxaliplatin. The term “pharmaceutically acceptable salt” refers to an addition salt that exists in conjunction with the acidic or basic portion of oxaliplatin. Such salts include the pharmaceutically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan. Pharmaceutically acceptable salts of an acid addition nature are formed when oxaliplatin and any of its intermediates containing a basic functionality are reacted with a pharmaceutically acceptable acid. Pharmaceutically acceptable acids commonly employed to form such acid addition salts include inorganic and organic acids. Pharmaceutically acceptable salts of a base addition nature are formed when oxaliplatin and any of its intermediates containing an acidic functionality are reacted with a pharmaceutically acceptable base. Pharmaceutically acceptable bases commonly employed to form base addition salts include organic and inorganic bases.

In addition to pharmaceutically acceptable salts, other salts are included in the present embodiments. They may serve as intermediates in the purification of compounds or in the preparation of other pharmaceutically acceptable salts, or are useful for identification, characterization or purification.

Mycophenolic acid, also known as 6-(1,3-Dihydro-4-hydroxy-6-methoxy-7-methyl-3-oxo-5-isobenzofuranyl)-4-methyl-2-(4-morpholinyl)ethyl ester hexenoic acid, is a compound that is an immunosuppressive agent. The chemical structure of mycophenolic acid is:

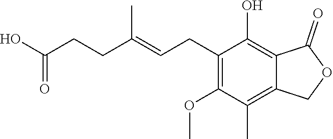 [SMILES: COc1c(C)c2c(c(O)c1C/C=C(\C)CCC(=O)O)C(=O)OC2]

As used herein, the term “mycophenolic acid” refers to mycophenolic acid base, pharmaceutically acceptable salts of mycophenolic acid, other salts of mycophenolic acid, metabolites of mycophenolic acid, and prodrugs of mycophenolic acid. The term “pharmaceutically acceptable salt” refers to an addition salt that exists in conjunction with the acidic or basic portion of mycophenolic acid. Such salts include the pharmaceutically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan. Pharmaceutically acceptable salts of an acid addition nature are formed when mycophenolic acid and any of its intermediates containing a basic functionality are reacted with a pharmaceutically acceptable acid. Pharmaceutically acceptable acids commonly employed to form such acid addition salts include inorganic and organic acids. Pharmaceutically acceptable salts of a base addition nature are formed when mycophenolic acid and any of its intermediates containing an acidic functionality are reacted with a pharmaceutically acceptable base. Pharmaceutically acceptable bases commonly employed to form base addition salts include organic and inorganic bases.

In addition to pharmaceutically acceptable salts, other salts are included in the present embodiments. They may serve as intermediates in the purification of compounds or in the preparation of other pharmaceutically acceptable salts, or are useful for identification, characterization or purification.

Mycophenolate is a compound that is an immunosuppressive agent. A prodrug of mycophenolate is mycophenolate mofetil, also known as 6-(1,3-Dihydro-4-hydroxy-6-methoxy-7-methyl-3-oxo-5-isobenzofuranyl)-4-methyl-2-(4-morpholinyl)ethyl ester hexenoic acid. The chemical structure of mycophenolate mofetil is:

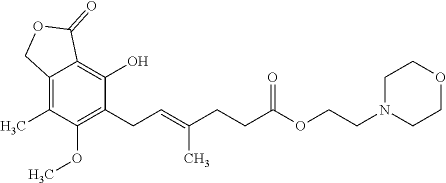 [SMILES: COc1c(C)c2c(c(O)c1C/C=C(\C)CCC(=O)OCCN1CCOCC1)C(=O)OC2]

As used herein, the term “mycophenolate” refers to mycophenolate base, pharmaceutically acceptable salts of mycophenolate mofetil, other salts of mycophenolate, metabolites of mycophenolate, and prodrugs of mycophenolate (e.g., mycophenolate mofetil). The term “pharmaceutically acceptable salt” refers to an addition salt that exists in conjunction with the acidic or basic portion of mycophenolate. Such salts include the pharmaceutically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan. Pharmaceutically acceptable salts of an acid addition nature are formed when mycophenolate and any of its intermediates containing a basic functionality are reacted with a pharmaceutically acceptable acid. Pharmaceutically acceptable acids commonly employed to form such acid addition salts include inorganic and organic acids. Pharmaceutically acceptable salts of a base addition nature are formed when mycophenolate and any of its intermediates containing an acidic functionality are reacted with a pharmaceutically acceptable base. Pharmaceutically acceptable bases commonly employed to form base addition salts include organic and inorganic bases.

In addition to pharmaceutically acceptable salts, other salts are included in the present embodiments. They may serve as intermediates in the purification of compounds or in the preparation of other pharmaceutically acceptable salts, or are useful for identification, characterization or purification.

In an embodiment, the first active ingredient is present at an amount between 1 μg/cm² to 1 mg/cm². In an embodiment, the first active ingredient is present at an amount between 1 μg/cm² to 100 g/cm². In an embodiment, the first active ingredient is present at an amount between 1 μg/cm² to 10 g/cm². In an embodiment, the first active ingredient is present at an amount between 10 μg/cm² to 20 g/cm². In an embodiment, the first active ingredient is present at an amount between 20 μg/cm² to 30 μg/cm². In an embodiment, the first active ingredient is present at an amount between 30 μg/cm² to 40 μg/cm². In an embodiment, the first active ingredient is present at an amount between 40 μg/cm² to 50 μg/cm². In an embodiment, the first active ingredient is present at an amount between 50 μg/cm² to 60 μg/cm². In an embodiment, the first active ingredient is present at an amount between 60 μg/cm² to 70 μg/cm². In an embodiment, the first active ingredient is present at an amount between 70 μg/cm² to 80 μg/cm². In an embodiment, the first active ingredient is present at an amount between 80 μg/cm² to 90 μg/cm². In an embodiment, the first active ingredient is present at an amount between 90 μg/cm² to 100 μg/cm². In an embodiment, the first active ingredient is present at an amount between 100 μg/cm² to 200 μg/cm². In an embodiment, the first active ingredient is present at an amount between 200 μg/cm² to 300 μg/cm². In an embodiment, the first active ingredient is present at an amount between 300 μg/cm² to 400 μg/cm². In an embodiment, the first active ingredient is present at an amount between 400 μg/cm² to 500 μg/cm². In an embodiment, the first active ingredient is present at an amount between 500 μg/cm² to 600 μg/cm². In an embodiment, the first active ingredient is present at an amount between 600 μg/cm² to 700 μg/cm². In an embodiment, the first active ingredient is present at an amount between 700 μg/cm² to 800 μg/cm². In an embodiment, the first active ingredient is present at an amount between 800 μg/cm² to 900 μg/cm². In an embodiment, the first active ingredient is present at an amount between 900 μg/cm² to 1 mg/cm².

In an embodiment, the second active ingredient is present at an amount between 10 μg/cm² to 10 mg/cm². In an embodiment, the second active ingredient is present at an amount between 10 μg/cm² to 1 mg/cm². In an embodiment, the second active ingredient is present at an amount between 10 μg/cm² to 100 μg/cm². In an embodiment, the second active ingredient is present at an amount between 10 μg/cm² to 20 μg/cm². In an embodiment, the second active ingredient is present at an amount between 20 μg/cm² to 30 μg/cm². In an embodiment, the second active ingredient is present at an amount between 30 μg/cm² to 40 μg/cm². In an embodiment, the second active ingredient is present at an amount between 40 μg/cm² to 50 μg/cm². In an embodiment, the second active ingredient is present at an amount between 50 μg/cm² to 60 μg/cm². In an embodiment, the second active ingredient is present at an amount between 60 μg/cm² to 70 μg/cm². In an embodiment, the second active ingredient is present at an amount between 70 μg/cm² to 80 μg/cm². In an embodiment, the second active ingredient is present at an amount between 80 μg/cm² to 90 μg/cm². In an embodiment, the second active ingredient is present at an amount between 90 μg/cm² to 100 μg/cm². In an embodiment, the second active ingredient is present at an amount between 100 μg/cm² to 150 μg/cm². In an embodiment, the second active ingredient is present at an amount between 100 μg/cm² to 150 μg/cm². In an embodiment, the second active ingredient is present at an amount between 150 μg/cm² to 200 μg/cm². In an embodiment, the second active ingredient is present at an amount between 200 μg/cm² to 250 μg/cm². In an embodiment, the second active ingredient is present at an amount between 250 μg/cm² to 300 μg/cm². In an embodiment, the second active ingredient is present at an amount between 300 μg/cm² to 350 μg/cm². In an embodiment, the second active ingredient is present at an amount between 350 μg/cm² to 400 μg/cm². In an embodiment, the second active ingredient is present at an amount between 400 μg/cm² to 450 μg/cm². In an embodiment, the second active ingredient is present at an amount between 450 μg/cm² to 500 μg/cm². In an embodiment, the second active ingredient is present at an amount between 500 μg/cm² to 550 μg/cm². In an embodiment, the second active ingredient is present at an amount between 550 μg/cm² to 600 μg/cm². In an embodiment, the second active ingredient is present at an amount between 600 μg/cm² to 650 μg/cm². In an embodiment, the second active ingredient is present at an amount between 650 μg/cm² to 700 μg/cm². In an embodiment, the second active ingredient is present at an amount between 700 μg/cm² to 750 μg/cm². In an embodiment, the second active ingredient is present at an amount between 750 μg/cm² to 800 μg/cm². In an embodiment, the second active ingredient is present at an amount between 800 μg/cm² to 850 μg/cm². In an embodiment, the second active ingredient is present at an amount between 850 μg/cm² to 900 μg/cm². In an embodiment, the second active ingredient is present at an amount between 900 μg/cm² to 950 μg/cm². In an embodiment, the second active ingredient is present at an amount between 950 μg/cm² to 1000 μg/cm². In an embodiment, the second active ingredient is present at an amount between 1 mg/cm² to 10 mg/cm². In an embodiment, the second active ingredient is present at an amount between 1 mg/cm² to 2 mg/cm². In an embodiment, the second active ingredient is present at an amount between 2 mg/cm² to 3 mg/cm². In an embodiment, the second active ingredient is present at an amount between 3 mg/cm² to 4 mg/cm². In an embodiment, the second active ingredient is present at an amount between 4 mg/cm² to 5 mg/cm². In an embodiment, the second active ingredient is present at an amount between 5 mg/cm² to 6 mg/cm². In an embodiment, the second active ingredient is present at an amount between 6 mg/cm² to 7 mg/cm². In an embodiment, the second active ingredient is present at an amount between 7 mg/cm² to 8 mg/cm². In an embodiment, the second active ingredient is present at an amount between 8 mg/cm² to 9 mg/cm². In an embodiment, the second active ingredient is present at an amount between 9 mg/cm² to 10 mg/cm².

In an embodiment, the pharmaceutical formulation comprises a first layer and a second layer. In an embodiment, the first layer is interposed between the support frame and the second layer. In an embodiment, the first layer comprises the first active ingredient and the second active ingredient. In an embodiment, the second layer comprises the first active ingredient and the second active ingredient.

In an embodiment, the first layer comprises the first active ingredient and the second active ingredient, wherein the first active ingredient and the second active ingredient are present at a first amount equal to the amount of the first active ingredient plus the amount of the second active ingredient in the first layer.

In an embodiment, the second layer comprises the first active ingredient and the second active ingredient, wherein the first active ingredient and the second active ingredient are present at a second amount equal to the amount of the first active ingredient plus the amount of the second active ingredient in the second layer.

In an embodiment, the ratio of first amount:second amount is 1:5. In an embodiment, the ratio of first amount:second amount is 1:4. In an embodiment, the ratio of first amount:second amount is 1:3. In an embodiment, the ratio of first amount:second amount is 1:2. In an embodiment, the ratio of first amount:second amount is 2:3. In an embodiment, the ratio of first amount:second amount is 1:1. In an embodiment, the ratio of first amount:second amount is 3:2. In an embodiment, the ratio of first amount:second amount is 2:1. In an embodiment, the ratio of first amount:second amount is 3:1. In an embodiment, the ratio of first amount:second amount is 4:1. In an embodiment, the ratio of first amount:second amount is 5:1.

In an embodiment, the first layer is configured to comprise two or more separate cells. In an embodiment, the first layer is configured to comprise a honeycomb pattern. In an embodiment, the second layer is configured to comprise two or more separate cells. In an embodiment, the second layer is configured to comprise a honeycomb pattern.

In an embodiment, the pharmaceutical formulation has a diameter of about 1 mm to about 1 cm. In an embodiment, the pharmaceutical formulation has a diameter of about 500 mm to about 1 cm. In an embodiment, the pharmaceutical formulation has a diameter of about 1 cm. In an embodiment, the pharmaceutical formulation has a diameter of about 2 cm. In an embodiment, the pharmaceutical formulation has a diameter of about 3 cm. In an embodiment, the pharmaceutical formulation has a diameter of about 4 cm. In an embodiment, the pharmaceutical formulation has a diameter of about 5 cm. In an embodiment, the pharmaceutical formulation has a diameter of about 6 cm. In an embodiment, the pharmaceutical formulation has a diameter of about 7 cm. In an embodiment, the pharmaceutical formulation has a diameter of about 8 cm. In an embodiment, the pharmaceutical formulation has a diameter of about 9 cm. In an embodiment, the pharmaceutical formulation has a diameter of about 10 cm. In an embodiment, the pharmaceutical formulation has a diameter between about 1 cm and about 10 cm. In an embodiment, the pharmaceutical formulation has a diameter between about 1 cm and about 5 cm.

In an illustrative aspect, a method of treating a disease in a subject is provided. The method comprises the step of administering to the subject a pharmaceutical formulation comprising i) a support frame, ii) one or more bioadhesive materials, iii) a first active ingredient, and iv) a second active ingredient for treatment of the disease. The previously described embodiments of the pharmaceutical formulation are also applicable to the method of treating a disease in a subject.

In an embodiment, the disease is a precancerous oral lesion. In an embodiment, the precancerous oral lesion is selected from the group consisting of leukoplakia, erythroplakia, erythroleukoplakia, proliferative verrucous leukoplakia, oral lichen planus, palatal lesions in reverse smokers, dyskeratosis congenital, and any combination thereof.

In an embodiment, the precancerous oral lesion is leukoplakia. In an embodiment, the precancerous oral lesion is erythroplakia. In an embodiment, the precancerous oral lesion is erythroleukoplakia. In an embodiment, the precancerous oral lesion is proliferative verrucous leukoplakia. In an embodiment, the precancerous oral lesion is oral lichen planus. In an embodiment, the precancerous oral lesion is palatal lesions in reverse smokers. In an embodiment, the precancerous oral lesion is dyskeratosis congenital.

In an embodiment, the disease is an oral potentially malignant disorder (OPMD). In an embodiment, the disease is an oral cancer. In an embodiment, the oral cancer is oral squamous cell carcinoma.

In an embodiment, the disease is associated with an oral dysplasia. In an embodiment, the oral dysplasia is a low-grade oral dysplasia. In an embodiment, the oral dysplasia is a medium grade oral dysplasia. In an embodiment, the oral dysplasia is a high-grade oral dysplasia.

In an embodiment, the administering comprises oral administration. In an embodiment, the oral administration is a sublingual administration. In an embodiment, the oral administration is a supralingual administration. In an embodiment, the oral administration is a buccal administration. In an embodiment, the oral administration is on the posterior tongue of the subject. In an embodiment, the oral administration is on the anterior tongue of the subject. In an embodiment, the oral administration is on the mouth floor of the subject.

In an embodiment, the administering is performed once per day. In an embodiment, the administering is performed two times per day. In an embodiment, the administering is performed three times per day. In an embodiment, the administering is performed once per week. In an embodiment, the administering is performed two times per week. In an embodiment, the administering is performed three times per week. In an embodiment, the administering is performed four times per week. In an embodiment, the administering is performed five times per week. In an embodiment, the administering is performed six times per week. In an embodiment, the administering is performed seven times per week.

In an embodiment, the subject is a mammal. In an embodiment, the subject is a human. In an embodiment, the first active ingredient is administered to the subject at a dose of about 0.1 to about 100 mg per kg of subject body weight. In an embodiment, the second active ingredient is administered to the subject at a dose of about 0.1 to about 100 mg per kg of subject body weight.

In an illustrative aspect, a process of making a pharmaceutical formulation comprising i) a support frame, ii) one or more bioadhesive materials, iii) a first active ingredient, and iv) a second active ingredient is provided. The process comprises the steps of: i) combining the one or more bioadhesive materials, the first active ingredient, the second active ingredient, and water to form a mixture; and ii) printing the mixture of step i) on the support frame to form the pharmaceutical formulation. The previously described embodiments of the pharmaceutical formulation are also applicable to the process of making a pharmaceutical formulation.

In an embodiment, step i) comprises dissolving the one or more bioadhesive materials, the first active ingredient, the second active ingredient in the water. In an embodiment, step i) further comprises blending the mixture using a mixer. In an embodiment, the mixture of step i) comprises a homogenous aqueous solution.

In an embodiment, the mixture of step i) is placed in a syringe prior to step ii). In an embodiment, the syringe is configured for printing the mixture of step i) on the support frame.

In an embodiment, step ii) comprises use of a three dimensional printer. In an embodiment, step ii) comprises controlling flow rate of the mixture via adjusting dispensing pressure of the mixture. In an embodiment, step ii) comprises controlling flow rate of the mixture via adjusting valve opening time. In an embodiment, step ii) comprises controlling flow rate of the mixture via dosing distance of the mixture. In an embodiment, step ii) comprises printing the mixture of step i) in two or more layers on the support frame.

In an embodiment, step ii) comprises printing the mixture of step i) in a first layer and a second layer on the support frame. In an embodiment, the first layer is interposed between the support frame and the second layer. In an embodiment, the first layer comprises the first active ingredient and the second active ingredient. In an embodiment, the second layer comprises the first active ingredient and the second active ingredient.

In an embodiment, the first layer comprises the first active ingredient and the second active ingredient, wherein the first active ingredient and the second active ingredient are present at a first amount equal to the amount of the first active ingredient plus the amount of the second active ingredient in the first layer.

In an embodiment, the second layer comprises the first active ingredient and the second active ingredient, wherein the first active ingredient and the second active ingredient are present at a second amount equal to the amount of the first active ingredient plus the amount of the second active ingredient in the second layer.

In an embodiment, the ratio of first amount:second amount is 1:5. In an embodiment, the ratio of first amount:second amount is 1:4. In an embodiment, the ratio of first amount:second amount is 1:3. In an embodiment, the ratio of first amount:second amount is 1:2. In an embodiment, the ratio of first amount:second amount is 2:3. In an embodiment, the ratio of first amount:second amount is 1:1. In an embodiment, the ratio of first amount:second amount is 3:2. In an embodiment, the ratio of first amount:second amount is 2:1. In an embodiment, the ratio of first amount:second amount is 3:1. In an embodiment, the ratio of first amount:second amount is 4:1. In an embodiment, the ratio of first amount:second amount is 5:1.

In an embodiment, the first layer is configured to comprise two or more separate cells. In an embodiment, the first layer is configured to comprise a honeycomb pattern. In an embodiment, the second layer is configured to comprise two or more separate cells. In an embodiment, the second layer is configured to comprise a honeycomb pattern.

In an illustrative aspect, a pharmaceutical formulation is provided, wherein the pharmaceutical formulation is prepared by the process steps described herein. The previously described embodiments of the pharmaceutical formulation are also applicable to the pharmaceutical formulation prepared by the process steps described herein.

The following numbered embodiments are contemplated and are non-limiting:
1. A pharmaceutical formulation comprising
i) a support frame,
ii) one or more bioadhesive materials,
iii) a first active ingredient, and
iv) a second active ingredient.
2. The pharmaceutical formulation of clause 1, any other suitable clause, or any combination of suitable clauses, wherein the pharmaceutical formulation is configured as a three-dimensional printed composition.
3. The pharmaceutical formulation of clause 1, any other suitable clause, or any combination of suitable clauses, wherein the support frame comprises plastic.
4. The pharmaceutical formulation of clause 1, any other suitable clause, or any combination of suitable clauses, wherein the support frame comprises one or more polymers.
5. The pharmaceutical formulation of clause 4, any other suitable clause, or any combination of suitable clauses, wherein the polymer is polyurethane.
6. The pharmaceutical formulation of clause 4, any other suitable clause, or any combination of suitable clauses, wherein the polymer is polyethylene.
7. The pharmaceutical formulation of clause 4, any other suitable clause, or any combination of suitable clauses, wherein the polymer is polyester.
8. The pharmaceutical formulation of clause 1, any other suitable clause, or any combination of suitable clauses, wherein the support frame comprises a polymeric film.
9. The pharmaceutical formulation of clause 8, any other suitable clause, or any combination of suitable clauses, wherein the polymeric film is a polyurethane film.
10. The pharmaceutical formulation of clause 8, any other suitable clause, or any combination of suitable clauses, wherein the polymeric film is a polyethylene film.
11. The pharmaceutical formulation of clause 8, any other suitable clause, or any combination of suitable clauses, wherein the polymeric film is a polyester film.
12. The pharmaceutical formulation of clause 1, any other suitable clause, or any combination of suitable clauses, wherein the support frame comprises a semi-permeable film.
13. The pharmaceutical formulation of clause 1, any other suitable clause, or any combination of suitable clauses, wherein the one or more bioadhesive materials are selected from the group consisting of polyacrylic acid (PAA), gelatin-modified dopamine (DOPA), carboxymethyl cellulose (CMC), polylactic acid, sodium carboxymethyl cellulose, carbopol, chitosan, PEG (Polyethylene glycol), sodium alginate, gelatin, pectin, Poly(vinyl alcohol), Poly(ethylene oxide), Poly(vinyl pyrrolidone), methylcellulose, methylethyl cellulose, gum tragacanth, soluble starch, and any combination thereof.
14. The pharmaceutical formulation of clause 1, any other suitable clause, or any combination of suitable clauses, wherein the one or more bioadhesive materials are selected from the group consisting of polyacrylic acid (PAA), gelatin-modified dopamine (DOPA), carboxymethyl cellulose (CMC), and any combination thereof.
15. The pharmaceutical formulation of clause 1, any other suitable clause, or any combination of suitable clauses, wherein the one or more bioadhesive materials comprises polyacrylic acid (PAA).
16. The pharmaceutical formulation of clause 1, any other suitable clause, or any combination of suitable clauses, wherein the one or more bioadhesive materials comprises gelatin-modified dopamine (DOPA).
17. The pharmaceutical formulation of clause 1, any other suitable clause, or any combination of suitable clauses, wherein the one or more bioadhesive materials comprises carboxymethyl cellulose (CMC).
18. The pharmaceutical formulation of clause 1, any other suitable clause, or any combination of suitable clauses, wherein the one or more bioadhesive materials comprises polylactic acid.
19. The pharmaceutical formulation of clause 1, any other suitable clause, or any combination of suitable clauses, wherein the one or more bioadhesive materials comprises sodium carboxymethyl cellulose.
20. The pharmaceutical formulation of clause 1, any other suitable clause, or any combination of suitable clauses, wherein the one or more bioadhesive materials comprises carbopol.
21. The pharmaceutical formulation of clause 1, any other suitable clause, or any combination of suitable clauses, wherein the one or more bioadhesive materials comprises chitosan.
22. The pharmaceutical formulation of clause 1, any other suitable clause, or any combination of suitable clauses, wherein the one or more bioadhesive materials comprises PEG (Polyethylene glycol).
23. The pharmaceutical formulation of clause 1, any other suitable clause, or any combination of suitable clauses, wherein the one or more bioadhesive materials comprises sodium alginate.
24. The pharmaceutical formulation of clause 1, any other suitable clause, or any combination of suitable clauses, wherein the one or more bioadhesive materials comprises gelatin.
25. The pharmaceutical formulation of clause 1, any other suitable clause, or any combination of suitable clauses, wherein the one or more bioadhesive materials comprises pectin.
26. The pharmaceutical formulation of clause 1, any other suitable clause, or any combination of suitable clauses, wherein the one or more bioadhesive materials comprises Poly(vinyl alcohol).
27. The pharmaceutical formulation of clause 1, any other suitable clause, or any combination of suitable clauses, wherein the one or more bioadhesive materials comprises Poly(ethylene oxide).
28. The pharmaceutical formulation of clause 1, any other suitable clause, or any combination of suitable clauses, wherein the one or more bioadhesive materials comprises Poly(vinyl pyrrolidone).
29. The pharmaceutical formulation of clause 1, any other suitable clause, or any combination of suitable clauses, wherein the one or more bioadhesive materials comprises methylcellulose.
30. The pharmaceutical formulation of clause 1, any other suitable clause, or any combination of suitable clauses, wherein the one or more bioadhesive materials comprises methylethyl cellulose.
31. The pharmaceutical formulation of clause 1, any other suitable clause, or any combination of suitable clauses, wherein the one or more bioadhesive materials comprises gum tragacanth.
32. The pharmaceutical formulation of clause 1, any other suitable clause, or any combination of suitable clauses, wherein the one or more bioadhesive materials comprises soluble starch.
33. The pharmaceutical formulation of clause 1, any other suitable clause, or any combination of suitable clauses, wherein the first active ingredient is oxaliplatin (OXP).
34. The pharmaceutical formulation of clause 1, any other suitable clause, or any combination of suitable clauses, wherein the first active ingredient is present at an amount between 1 μg/cm² to 1 mg/cm².
35. The pharmaceutical formulation of clause 1, any other suitable clause, or any combination of suitable clauses, wherein the first active ingredient is present at an amount between 1 μg/cm² to 100 μg/cm².
36. The pharmaceutical formulation of clause 1, any other suitable clause, or any combination of suitable clauses, wherein the first active ingredient is present at an amount between 1 μg/cm² to 10 μg/cm².
37. The pharmaceutical formulation of clause 1, any other suitable clause, or any combination of suitable clauses, wherein the first active ingredient is present at an amount between 10 μg/cm² to 20 μg/cm².
38. The pharmaceutical formulation of clause 1, any other suitable clause, or any combination of suitable clauses, wherein the first active ingredient is present at an amount between 20 μg/cm² to 30 μg/cm².
39. The pharmaceutical formulation of clause 1, any other suitable clause, or any combination of suitable clauses, wherein the first active ingredient is present at an amount between 30 μg/cm² to 40 μg/cm².
40. The pharmaceutical formulation of clause 1, any other suitable clause, or any combination of suitable clauses, wherein the first active ingredient is present at an amount between 40 μg/cm² to 50 μg/cm².
41. The pharmaceutical formulation of clause 1, any other suitable clause, or any combination of suitable clauses, wherein the first active ingredient is present at an amount between 50 μg/cm² to 60 μg/cm².
42. The pharmaceutical formulation of clause 1, any other suitable clause, or any combination of suitable clauses, wherein the first active ingredient is present at an amount between 60 μg/cm² to 70 μg/cm².
43. The pharmaceutical formulation of clause 1, any other suitable clause, or any combination of suitable clauses, wherein the first active ingredient is present at an amount between 70 μg/cm² to 80 μg/cm².
44. The pharmaceutical formulation of clause 1, any other suitable clause, or any combination of suitable clauses, wherein the first active ingredient is present at an amount between 80 μg/cm² to 90 μg/cm².
45. The pharmaceutical formulation of clause 1, any other suitable clause, or any combination of suitable clauses, wherein the first active ingredient is present at an amount between 90 μg/cm² to 100 μg/cm².
46. The pharmaceutical formulation of clause 1, any other suitable clause, or any combination of suitable clauses, wherein the first active ingredient is present at an amount between 100 μg/cm² to 200 μg/cm².
47. The pharmaceutical formulation of clause 1, any other suitable clause, or any combination of suitable clauses, wherein the first active ingredient is present at an amount between 200 μg/cm² to 300 μg/cm².
48. The pharmaceutical formulation of clause 1, any other suitable clause, or any combination of suitable clauses, wherein the first active ingredient is present at an amount between 300 μg/cm² to 400 μg/cm².
49. The pharmaceutical formulation of clause 1, any other suitable clause, or any combination of suitable clauses, wherein the first active ingredient is present at an amount between 400 μg/cm² to 500 μg/cm².
50. The pharmaceutical formulation of clause 1, any other suitable clause, or any combination of suitable clauses, wherein the first active ingredient is present at an amount between 500 μg/cm² to 600 μg/cm².
51. The pharmaceutical formulation of clause 1, any other suitable clause, or any combination of suitable clauses, wherein the first active ingredient is present at an amount between 600 μg/cm² to 700 μg/cm².
52. The pharmaceutical formulation of clause 1, any other suitable clause, or any combination of suitable clauses, wherein the first active ingredient is present at an amount between 700 μg/cm² to 800 μg/cm².
53. The pharmaceutical formulation of clause 1, any other suitable clause, or any combination of suitable clauses, wherein the first active ingredient is present at an amount between 800 μg/cm² to 900 μg/cm².
54. The pharmaceutical formulation of clause 1, any other suitable clause, or any combination of suitable clauses, wherein the first active ingredient is present at an amount between 900 μg/cm² to 1 mg/cm².
55. The pharmaceutical formulation of clause 1, any other suitable clause, or any combination of suitable clauses, wherein the second active ingredient is mycophenolic acid (MPA).
56. The pharmaceutical formulation of clause 1, any other suitable clause, or any combination of suitable clauses, wherein the second active ingredient is mycophenolate (MPS).
57. The pharmaceutical formulation of clause 1, any other suitable clause, or any combination of suitable clauses, wherein the second active ingredient is mycophenolate motefil.
58. The pharmaceutical formulation of clause 1, any other suitable clause, or any combination of suitable clauses, wherein the second active ingredient is present at an amount between 10 μg/cm² to 10 mg/cm².
59. The pharmaceutical formulation of clause 1, any other suitable clause, or any combination of suitable clauses, wherein the second active ingredient is present at an amount between 10 μg/cm² to 1 mg/cm².
60. The pharmaceutical formulation of clause 1, any other suitable clause, or any combination of suitable clauses, wherein the second active ingredient is present at an amount between 10 μg/cm² to 100 μg/cm².
61. The pharmaceutical formulation of clause 1, any other suitable clause, or any combination of suitable clauses, wherein the second active ingredient is present at an amount between 10 μg/cm² to 20 μg/cm².
62. The pharmaceutical formulation of clause 1, any other suitable clause, or any combination of suitable clauses, wherein the second active ingredient is present at an amount between 20 μg/cm² to 30 μg/cm².
63. The pharmaceutical formulation of clause 1, any other suitable clause, or any combination of suitable clauses, wherein the second active ingredient is present at an amount between 30 μg/cm² to 40 μg/cm².
64. The pharmaceutical formulation of clause 1, any other suitable clause, or any combination of suitable clauses, wherein the second active ingredient is present at an amount between 40 μg/cm² to 50 μg/cm².
65. The pharmaceutical formulation of clause 1, any other suitable clause, or any combination of suitable clauses, wherein the second active ingredient is present at an amount between 50 μg/cm² to 60 μg/cm².
66. The pharmaceutical formulation of clause 1, any other suitable clause, or any combination of suitable clauses, wherein the second active ingredient is present at an amount between 60 μg/cm² to 70 μg/cm².
67. The pharmaceutical formulation of clause 1, any other suitable clause, or any combination of suitable clauses, wherein the second active ingredient is present at an amount between 70 μg/cm² to 80 μg/cm².
68. The pharmaceutical formulation of clause 1, any other suitable clause, or any combination of suitable clauses, wherein the second active ingredient is present at an amount between 80 μg/cm² to 90 μg/cm².
69. The pharmaceutical formulation of clause 1, any other suitable clause, or any combination of suitable clauses, wherein the second active ingredient is present at an amount between 90 μg/cm² to 100 μg/cm².
70. The pharmaceutical formulation of clause 1, any other suitable clause, or any combination of suitable clauses, wherein the second active ingredient is present at an amount between 100 μg/cm² to 150 μg/cm².
71. The pharmaceutical formulation of clause 1, any other suitable clause, or any combination of suitable clauses, wherein the second active ingredient is present at an amount between 100 μg/cm² to 150 μg/cm².
72. The pharmaceutical formulation of clause 1, any other suitable clause, or any combination of suitable clauses, wherein the second active ingredient is present at an amount between 150 μg/cm² to 200 μg/cm².
73. The pharmaceutical formulation of clause 1, any other suitable clause, or any combination of suitable clauses, wherein the second active ingredient is present at an amount between 200 μg/cm² to 250 μg/cm².
74. The pharmaceutical formulation of clause 1, any other suitable clause, or any combination of suitable clauses, wherein the second active ingredient is present at an amount between 250 μg/cm² to 300 μg/cm².
75. The pharmaceutical formulation of clause 1, any other suitable clause, or any combination of suitable clauses, wherein the second active ingredient is present at an amount between 300 μg/cm² to 350 μg/cm².
76. The pharmaceutical formulation of clause 1, any other suitable clause, or any combination of suitable clauses, wherein the second active ingredient is present at an amount between 350 μg/cm² to 400 μg/cm².
77. The pharmaceutical formulation of clause 1, any other suitable clause, or any combination of suitable clauses, wherein the second active ingredient is present at an amount between 400 μg/cm² to 450 μg/cm².
78. The pharmaceutical formulation of clause 1, any other suitable clause, or any combination of suitable clauses, wherein the second active ingredient is present at an amount between 450 μg/cm² to 500 μg/cm².
79. The pharmaceutical formulation of clause 1, any other suitable clause, or any combination of suitable clauses, wherein the second active ingredient is present at an amount between 500 μg/cm² to 550 μg/cm².
80. The pharmaceutical formulation of clause 1, any other suitable clause, or any combination of suitable clauses, wherein the second active ingredient is present at an amount between 550 μg/cm² to 600 μg/cm².
81. The pharmaceutical formulation of clause 1, any other suitable clause, or any combination of suitable clauses, wherein the second active ingredient is present at an amount between 600 μg/cm² to 650 μg/cm².
82. The pharmaceutical formulation of clause 1, any other suitable clause, or any combination of suitable clauses, wherein the second active ingredient is present at an amount between 650 μg/cm² to 700 μg/cm².
83. The pharmaceutical formulation of clause 1, any other suitable clause, or any combination of suitable clauses, wherein the second active ingredient is present at an amount between 700 μg/cm² to 750 μg/cm².
84. The pharmaceutical formulation of clause 1, any other suitable clause, or any combination of suitable clauses, wherein the second active ingredient is present at an amount between 750 μg/cm² to 800 μg/cm².
85. The pharmaceutical formulation of clause 1, any other suitable clause, or any combination of suitable clauses, wherein the second active ingredient is present at an amount between 800 μg/cm² to 850 μg/cm².
86. The pharmaceutical formulation of clause 1, any other suitable clause, or any combination of suitable clauses, wherein the second active ingredient is present at an amount between 850 μg/cm² to 900 μg/cm².
87. The pharmaceutical formulation of clause 1, any other suitable clause, or any combination of suitable clauses, wherein the second active ingredient is present at an amount between 900 μg/cm² to 950 μg/cm².
88. The pharmaceutical formulation of clause 1, any other suitable clause, or any combination of suitable clauses, wherein the second active ingredient is present at an amount between 950 μg/cm² to 1000 μg/cm².
89. The pharmaceutical formulation of clause 1, any other suitable clause, or any combination of suitable clauses, wherein the second active ingredient is present at an amount between 1 mg/cm² to 10 mg/cm².
90. The pharmaceutical formulation of clause 1, any other suitable clause, or any combination of suitable clauses, wherein the second active ingredient is present at an amount between 1 mg/cm² to 2 mg/cm².
91. The pharmaceutical formulation of clause 1, any other suitable clause, or any combination of suitable clauses, wherein the second active ingredient is present at an amount between 2 mg/cm² to 3 mg/cm².
92. The pharmaceutical formulation of clause 1, any other suitable clause, or any combination of suitable clauses, wherein the second active ingredient is present at an amount between 3 mg/cm² to 4 mg/cm².
93. The pharmaceutical formulation of clause 1, any other suitable clause, or any combination of suitable clauses, wherein the second active ingredient is present at an amount between 4 mg/cm² to 5 mg/cm².
94. The pharmaceutical formulation of clause 1, any other suitable clause, or any combination of suitable clauses, wherein the second active ingredient is present at an amount between 5 mg/cm² to 6 mg/cm².
95. The pharmaceutical formulation of clause 1, any other suitable clause, or any combination of suitable clauses, wherein the second active ingredient is present at an amount between 6 mg/cm² to 7 mg/cm².
96. The pharmaceutical formulation of clause 1, any other suitable clause, or any combination of suitable clauses, wherein the second active ingredient is present at an amount between 7 mg/cm² to 8 mg/cm².
97. The pharmaceutical formulation of clause 1, any other suitable clause, or any combination of suitable clauses, wherein the second active ingredient is present at an amount between 8 mg/cm² to 9 mg/cm².
98. The pharmaceutical formulation of clause 1, any other suitable clause, or any combination of suitable clauses, wherein the second active ingredient is present at an amount between 9 mg/cm² to 10 mg/cm².
99. The pharmaceutical formulation of clause 1, any other suitable clause, or any combination of suitable clauses, wherein the pharmaceutical formulation comprises a first layer and a second layer.
100. The pharmaceutical formulation of clause 99, any other suitable clause, or any combination of suitable clauses, wherein the first layer is interposed between the support frame and the second layer.
101. The pharmaceutical formulation of clause 99, any other suitable clause, or any combination of suitable clauses, wherein the first layer comprises the first active ingredient and the second active ingredient.
102. The pharmaceutical formulation of clause 99, any other suitable clause, or any combination of suitable clauses, wherein the second layer comprises the first active ingredient and the second active ingredient.
103. The pharmaceutical formulation of clause 99, any other suitable clause, or any combination of suitable clauses, wherein the first layer comprises the first active ingredient and the second active ingredient, wherein the first active ingredient and the second active ingredient are present at a first amount equal to the amount of the first active ingredient plus the amount of the second active ingredient in the first layer.
104. The pharmaceutical formulation of clause 99, any other suitable clause, or any combination of suitable clauses, wherein the second layer comprises the first active ingredient and the second active ingredient, wherein the first active ingredient and the second active ingredient are present at a second amount equal to the amount of the first active ingredient plus the amount of the second active ingredient in the second layer.
105. The pharmaceutical formulation of clause 104, any other suitable clause, or any combination of suitable clauses, wherein the ratio of first amount:second amount is 1:5.
106. The pharmaceutical formulation of clause 104, any other suitable clause, or any combination of suitable clauses, wherein the ratio of first amount:second amount is 1:4.
107. The pharmaceutical formulation of clause 104, any other suitable clause, or any combination of suitable clauses, wherein the ratio of first amount:second amount is 1:3.
108. The pharmaceutical formulation of clause 104, any other suitable clause, or any combination of suitable clauses, wherein the ratio of first amount:second amount is 1:2.
109. The pharmaceutical formulation of clause 104, any other suitable clause, or any combination of suitable clauses, wherein the ratio of first amount:second amount is 2:3.
110. The pharmaceutical formulation of clause 104, any other suitable clause, or any combination of suitable clauses, wherein the ratio of first amount:second amount is 1:1.
111. The pharmaceutical formulation of clause 104, any other suitable clause, or any combination of suitable clauses, wherein the ratio of first amount:second amount is 3:2.
112. The pharmaceutical formulation of clause 104, any other suitable clause, or any combination of suitable clauses, wherein the ratio of first amount:second amount is 2:1.
113. The pharmaceutical formulation of clause 104, any other suitable clause, or any combination of suitable clauses, wherein the ratio of first amount:second amount is 3:1.
114. The pharmaceutical formulation of clause 104, any other suitable clause, or any combination of suitable clauses, wherein the ratio of first amount:second amount is 4:1.
115. The pharmaceutical formulation of clause 104, any other suitable clause, or any combination of suitable clauses, wherein the ratio of first amount:second amount is 5:1.
116. The pharmaceutical formulation of clause 99, any other suitable clause, or any combination of suitable clauses, wherein the first layer is configured to comprise two or more separate cells.
117. The pharmaceutical formulation of clause 99, any other suitable clause, or any combination of suitable clauses, wherein the first layer is configured to comprise a honeycomb pattern.
118. The pharmaceutical formulation of clause 99, any other suitable clause, or any combination of suitable clauses, wherein the second layer is configured to comprise two or more separate cells.
119. The pharmaceutical formulation of clause 99, any other suitable clause, or any combination of suitable clauses, wherein the second layer is configured to comprise a honeycomb pattern.
120. The pharmaceutical formulation of clause 1, any other suitable clause, or any combination of suitable clauses, wherein the pharmaceutical formulation has a diameter of about 1 mm to about 1 cm.
121. The pharmaceutical formulation of clause 1, any other suitable clause, or any combination of suitable clauses, wherein the pharmaceutical formulation has a diameter of about 500 mm to about 1 cm.
122. The pharmaceutical formulation of clause 1, any other suitable clause, or any combination of suitable clauses, wherein the pharmaceutical formulation has a diameter of about 1 cm.
123. The pharmaceutical formulation of clause 1, any other suitable clause, or any combination of suitable clauses, wherein the pharmaceutical formulation has a diameter of about 2 cm.
124. The pharmaceutical formulation of clause 1, any other suitable clause, or any combination of suitable clauses, wherein the pharmaceutical formulation has a diameter of about 3 cm.
125. The pharmaceutical formulation of clause 1, any other suitable clause, or any combination of suitable clauses, wherein the pharmaceutical formulation has a diameter of about 4 cm.
126. The pharmaceutical formulation of clause 1, any other suitable clause, or any combination of suitable clauses, wherein the pharmaceutical formulation has a diameter of about 5 cm.
127. The pharmaceutical formulation of clause 1, any other suitable clause, or any combination of suitable clauses, wherein the pharmaceutical formulation has a diameter of about 6 cm.
128. The pharmaceutical formulation of clause 1, any other suitable clause, or any combination of suitable clauses, wherein the pharmaceutical formulation has a diameter of about 7 cm.
129. The pharmaceutical formulation of clause 1, any other suitable clause, or any combination of suitable clauses, wherein the pharmaceutical formulation has a diameter of about 8 cm.
130. The pharmaceutical formulation of clause 1, any other suitable clause, or any combination of suitable clauses, wherein the pharmaceutical formulation has a diameter of about 9 cm.
131. The pharmaceutical formulation of clause 1, any other suitable clause, or any combination of suitable clauses, wherein the pharmaceutical formulation has a diameter of about 10 cm.
132. The pharmaceutical formulation of clause 1, any other suitable clause, or any combination of suitable clauses, wherein the pharmaceutical formulation has a diameter between about 1 cm and about 10 cm.
133. The pharmaceutical formulation of clause 1, any other suitable clause, or any combination of suitable clauses, wherein the pharmaceutical formulation has a diameter between about 1 cm and about 5 cm.
134. A method of treating a disease in a subject, said method comprising the step of administering to the subject a pharmaceutical formulation comprising i) a support frame, ii) one or more bioadhesive materials, iii) a first active ingredient, and iv) a second active ingredient for treatment of the disease.
135. The method of clause 134, any other suitable clause, or any combination of suitable clauses, wherein the disease is a precancerous oral lesion.
136. The method of clause 135, any other suitable clause, or any combination of suitable clauses, wherein the precancerous oral lesion is selected from the group consisting of leukoplakia, erythroplakia, erythroleukoplakia, proliferative verrucous leukoplakia, oral lichen planus, palatal lesions in reverse smokers, dyskeratosis congenital, and any combination thereof.
137. The method of clause 135, any other suitable clause, or any combination of suitable clauses, wherein the precancerous oral lesion is leukoplakia.
138. The method of clause 135, any other suitable clause, or any combination of suitable clauses, wherein the precancerous oral lesion is erythroplakia.
139. The method of clause 135, any other suitable clause, or any combination of suitable clauses, wherein the precancerous oral lesion is erythroleukoplakia.
140. The method of clause 135, any other suitable clause, or any combination of suitable clauses, wherein the precancerous oral lesion is proliferative verrucous leukoplakia.
141. The method of clause 135, any other suitable clause, or any combination of suitable clauses, wherein the precancerous oral lesion is oral lichen planus.
142. The method of clause 135, any other suitable clause, or any combination of suitable clauses, wherein the precancerous oral lesion is palatal lesions in reverse smokers.
143. The method of clause 135, any other suitable clause, or any combination of suitable clauses, wherein the precancerous oral lesion is dyskeratosis congenital.
144. The method of clause 134, any other suitable clause, or any combination of suitable clauses, wherein the disease is an oral potentially malignant disorder (OPMD).
145. The method of clause 134, any other suitable clause, or any combination of suitable clauses, wherein the disease is an oral cancer.
146. The method of clause 145, any other suitable clause, or any combination of suitable clauses, wherein the oral cancer is oral squamous cell carcinoma.
147. The method of clause 134, any other suitable clause, or any combination of suitable clauses, wherein the disease is associated with an oral dysplasia.
148. The method of clause 147, any other suitable clause, or any combination of suitable clauses, wherein the oral dysplasia is a low-grade oral dysplasia.
149. The method of clause 147, any other suitable clause, or any combination of suitable clauses, wherein the oral dysplasia is a medium grade oral dysplasia.
150. The method of clause 147, any other suitable clause, or any combination of suitable clauses, wherein the oral dysplasia is a high-grade oral dysplasia.
151. The method of clause 134, any other suitable clause, or any combination of suitable clauses, wherein the administering comprises oral administration.
152. The method of clause 151, any other suitable clause, or any combination of suitable clauses, wherein the oral administration is a sublingual administration.
153. The method of clause 151, any other suitable clause, or any combination of suitable clauses, wherein the oral administration is a supralingual administration.
154. The method of clause 151, any other suitable clause, or any combination of suitable clauses, wherein the oral administration is a buccal administration.
155. The method of clause 151, any other suitable clause, or any combination of suitable clauses, wherein the oral administration is on the posterior tongue of the subject.
156. The method of clause 151, any other suitable clause, or any combination of suitable clauses, wherein the oral administration is on the anterior tongue of the subject.
157. The method of clause 151, any other suitable clause, or any combination of suitable clauses, wherein the oral administration is on the mouth floor of the subject.
158. The method of clause 134, any other suitable clause, or any combination of suitable clauses, wherein the administering is performed once per day.
159. The method of clause 134, any other suitable clause, or any combination of suitable clauses, wherein the administering is performed two times per day.
160. The method of clause 134, any other suitable clause, or any combination of suitable clauses, wherein the administering is performed three times per day.
161. The method of clause 134, any other suitable clause, or any combination of suitable clauses, wherein the administering is performed once per week.
162. The method of clause 134, any other suitable clause, or any combination of suitable clauses, wherein the administering is performed two times per week.
163. The method of clause 134, any other suitable clause, or any combination of suitable clauses, wherein the administering is performed three times per week.
164. The method of clause 134, any other suitable clause, or any combination of suitable clauses, wherein the administering is performed four times per week.
165. The method of clause 134, any other suitable clause, or any combination of suitable clauses, wherein the administering is performed five times per week.
166. The method of clause 134, any other suitable clause, or any combination of suitable clauses, wherein the administering is performed six times per week.
167. The method of clause 134, any other suitable clause, or any combination of suitable clauses, wherein the administering is performed seven times per week.
168. The method of clause 134, any other suitable clause, or any combination of suitable clauses, wherein the subject is a mammal.
169. The method of clause 134, any other suitable clause, or any combination of suitable clauses, wherein the subject is a human.
170. The method of clause 134, any other suitable clause, or any combination of suitable clauses, wherein the first active ingredient is administered to the subject at a dose of about 0.1 to about 100 mg per kg of subject body weight.
171. The method of clause 134, any other suitable clause, or any combination of suitable clauses, wherein the second active ingredient is administered to the subject at a dose of about 0.1 to about 100 mg per kg of subject body weight.
172. A process of making a pharmaceutical formulation comprising i) a support frame, ii) one or more bioadhesive materials, iii) a first active ingredient, and iv) a second active ingredient, said process comprising the steps of:
i) combining the one or more bioadhesive materials, the first active ingredient, the second active ingredient, and water to form a mixture;
ii) printing the mixture of step i) on the support frame to form the pharmaceutical formulation.
173. The process of clause 172, any other suitable clause, or any combination of suitable clauses, wherein step i) comprises dissolving the one or more bioadhesive materials, the first active ingredient, the second active ingredient in the water.
174. The process of clause 172, any other suitable clause, or any combination of suitable clauses, wherein step i) further comprises blending the mixture using a mixer.
175. The process of clause 172, any other suitable clause, or any combination of suitable clauses, wherein the mixture of step i) comprises a homogenous aqueous solution.
176. The process of clause 172, any other suitable clause, or any combination of suitable clauses, wherein the mixture of step i) is placed in a syringe prior to step ii).
177. The process of clause 176, any other suitable clause, or any combination of suitable clauses, wherein the syringe is configured for printing the mixture of step i) on the support frame.
178. The process of clause 172, any other suitable clause, or any combination of suitable clauses, wherein step ii) comprises use of a three dimensional printer.
179. The process of clause 172, any other suitable clause, or any combination of suitable clauses, wherein step ii) comprises controlling flow rate of the mixture via adjusting dispensing pressure of the mixture.
180. The process of clause 172, any other suitable clause, or any combination of suitable clauses, wherein step ii) comprises controlling flow rate of the mixture via adjusting valve opening time.
181. The process of clause 172, any other suitable clause, or any combination of suitable clauses, wherein step ii) comprises controlling flow rate of the mixture via dosing distance of the mixture.
182. The process of clause 172, any other suitable clause, or any combination of suitable clauses, wherein step ii) comprises printing the mixture of step i) in two or more layers on the support frame.
183. The process of clause 172, any other suitable clause, or any combination of suitable clauses, wherein step ii) comprises printing the mixture of step i) in a first layer and a second layer on the support frame.
184. The process of clause 183, any other suitable clause, or any combination of suitable clauses, wherein the first layer is interposed between the support frame and the second layer.
185. The process of clause 183, any other suitable clause, or any combination of suitable clauses, wherein the first layer comprises the first active ingredient and the second active ingredient.
186. The process of clause 183, any other suitable clause, or any combination of suitable clauses, wherein the second layer comprises the first active ingredient and the second active ingredient.
187. The process of clause 183, any other suitable clause, or any combination of suitable clauses, wherein the first layer comprises the first active ingredient and the second active ingredient, wherein the first active ingredient and the second active ingredient are present at a first amount equal to the amount of the first active ingredient plus the amount of the second active ingredient in the first layer.
188. The process of clause 183, any other suitable clause, or any combination of suitable clauses, wherein the second layer comprises the first active ingredient and the second active ingredient, wherein the first active ingredient and the second active ingredient are present at a second amount equal to the amount of the first active ingredient plus the amount of the second active ingredient in the second layer.
189. The process of clause 188, any other suitable clause, or any combination of suitable clauses, wherein the ratio of first amount:second amount is 1:5.
190. The process of clause 188, any other suitable clause, or any combination of suitable clauses, wherein the ratio of first amount:second amount is 1:4.
191. The process of clause 188, any other suitable clause, or any combination of suitable clauses, wherein the ratio of first amount:second amount is 1:3.
192. The process of clause 188, any other suitable clause, or any combination of suitable clauses, wherein the ratio of first amount:second amount is 1:2.
193. The process of clause 188, any other suitable clause, or any combination of suitable clauses, wherein the ratio of first amount:second amount is 2:3.
194. The process of clause 188, any other suitable clause, or any combination of suitable clauses, wherein the ratio of first amount:second amount is 1:1.
195. The process of clause 188, any other suitable clause, or any combination of suitable clauses, wherein the ratio of first amount:second amount is 3:2.
196. The process of clause 188, any other suitable clause, or any combination of suitable clauses, wherein the ratio of first amount:second amount is 2:1.
197. The process of clause 188, any other suitable clause, or any combination of suitable clauses, wherein the ratio of first amount:second amount is 3:1.
198. The process of clause 188, any other suitable clause, or any combination of suitable clauses, wherein the ratio of first amount:second amount is 4:1.
199. The process of clause 188, any other suitable clause, or any combination of suitable clauses, wherein the ratio of first amount:second amount is 5:1.
200. The process of clause 183, any other suitable clause, or any combination of suitable clauses, wherein the first layer is configured to comprise two or more separate cells.
201. The process of clause 183, any other suitable clause, or any combination of suitable clauses, wherein the first layer is configured to comprise a honeycomb pattern.
202. The process of clause 183, any other suitable clause, or any combination of suitable clauses, wherein the second layer is configured to comprise two or more separate cells.
203. The process of clause 183, any other suitable clause, or any combination of suitable clauses, wherein the second layer is configured to comprise a honeycomb pattern.
204. A pharmaceutical formulation prepared by the process of any one of clauses 172 to 203.
205. The pharmaceutical formulation of clause 204, wherein the pharmaceutical formulation corresponds to the pharmaceutical formulation of any one of clauses 1 to 133.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Exemplary Process for Making Pharmaceutical Formulation

The instant example provides an exemplary process for making the pharmaceutical formulation described herein. The pharmaceutical formulation of this example is designed to contain multiple layers comprising bioadhesive materials and two active ingredients.

The pharmaceutical formulation comprises a support frame to prevent water leakage without affecting oxygen permeation. For the instant example, Tegaderm™ transparent film was utilized as the support frame.

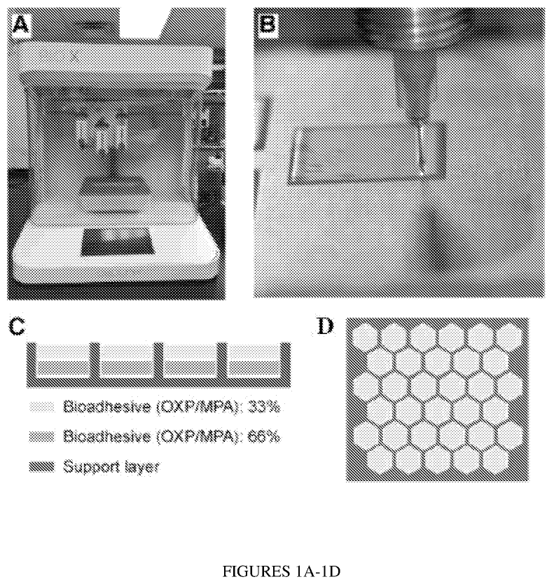

Fabrication of the pharmaceutical formulation was carried out using a CELLINK_BIO_X_3 D printer (see FIG. 1A). Bioadhesive materials include polyacrylic acid (PAA, Carbopol® 974 polymer), gelatin-modified dopamine (DOPA), and carboxymethyl cellulose (CMC) were used as well as the active ingredients oxaliplatin (“OXP”; 102544-196, Sigma) and mycophenolate (“MPS”; 1448989, Sigma).

To fabricate a single-layered pharmaceutical formulation comprising OXP and MPS, the bioadhesive materials (e.g., 0.1 gm of polyacrylic acid-974 (PAA-974), 0.1 gm of DOPA, and 0.2 gm of CMC), were combined with 1.5 mg of OXP and 20 mg of MPS then dissolved in 10 mL of distilled water. This mixture was blended in a Thinky Mixer (ARE-310, THINKY USA Inc.) for about 40 minutes to form a homogenous aqueous solution.

The homogenous aqueous solution was then added to a syringe with a 300 μm diameter nozzle and printed in a square sheet (30 mm×30 mm×20 μm, containing 48.56 μg of OXP and 648 μg of MPS) directly on the support frame (see FIG. 1B). During the printing process, the flow rate was controlled by adjusting the dispensing pressure, the valve opening time, and the dosing distance. The width of the printed lines was controlled by adjusting the printing speed and the parameters mentioned above.

To fabricate a double-layered pharmaceutical formulation comprising OXP and MPS (30 mm×30 mm×20 μm) with a top-to-bottom active ingredient ratio of 1-to-2 (see FIG. 1C). Bioadhesive materials were prepared as described above, evenly divided into two parts, and then combined with OXP and MPS in the amounts of 0.5 mg/6.67 mg (OXP/MPS) for the top layer and 1.0 mg/13.33 mg for the bottom layer. Square sheets (30 mm×30 mm) were printed directly onto the supportive frame layer by layer. After a drying time of 24 hours at 37° C. after each layer was printed, the thickness of the layers was reduced to 20 μm. For further evaluation, the pharmaceutical formulation was punched into circular compositions of different diameters using a Miltex biopsy punch with plunger (4MD Medical, Lakewood, New Jersey). In some embodiments, the pharmaceutical formulation can be designed in a honeycomb pattern printed on the support frame, wherein the honeycomb pattern comprises several separate cells (see FIG. 1D).

### Example 2

### Exemplary Processes for Making Various Pharmaceutical Formulation

The instant example provides different exemplary processes for developing various iterations of pharmaceutical formulations.

### Version 1

A pharmaceutical formulation was made by embedding 3 mg of OXP and 20 mg of MPS into a single-layered square sheet (30 mm×30 mm×20 μm). Discs of 5-mm in radius were punched and immersed in distilled water for 30 minutes to 6 hours.

The amounts of OXP and MPS were measured by UPLC and calculated as percentages of the theoretical loaded amounts within the disc (see FIG. 2A and FIG. 2B, respectively). Each data point represents sample size of three.

### Version 2

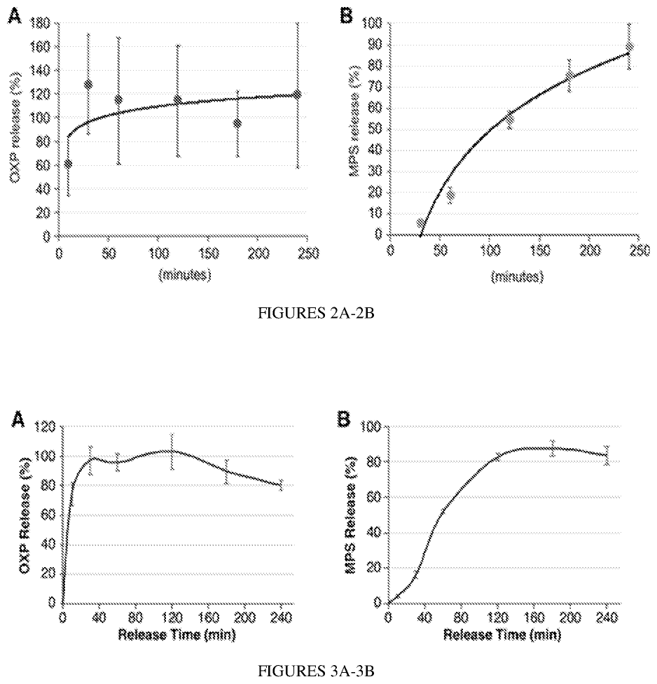

For the active ingredient release profile, MPS was shown to display a sustained release from 0 to 3 hours, reaching a plateau at 87.1% of the extractable amount and 66.0% of the loaded amount after 3 hours (see FIG. 3A). However, OXP exhibited an abrupt release within 10-30 minutes, reaching a plateau at 89.3% of the extractable amount and only 9.4% of the loaded amount after 30 minutes (FIG. 3B).

### Version 3

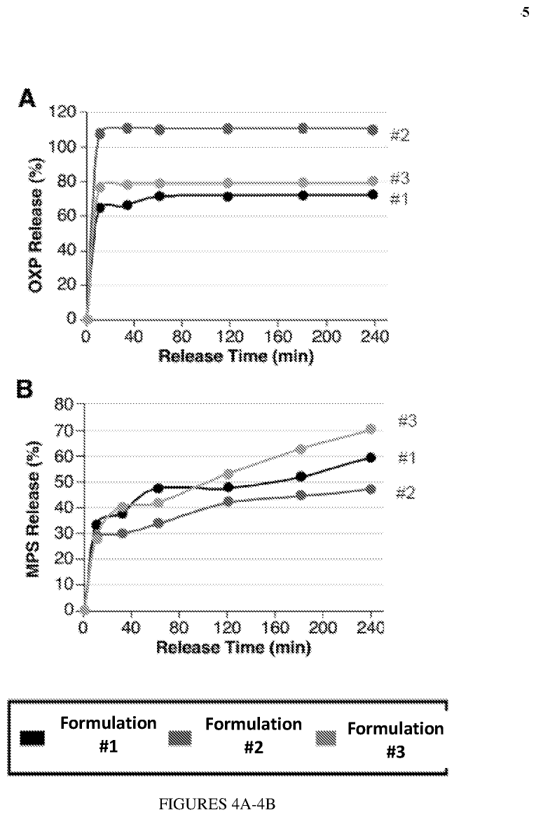

To further optimize the pharmaceutical formulation and address the ineffective release of OXP, a third-generation formulation was designed to improve the released fraction of OXP. Results showed that the release efficiency of OXP from this pharmaceutical formulation is much improved, reaching an average of 87% of the loaded amount, as compared to the previous 9.4% from the second-generation formulation (FIG. 4A).

The release efficiency of MPS remained at 59% of the total loaded amount, as compared to the previous 66% from the second-generation formulation (FIG. 4B). However, this adjustment in the formulation still demonstrated a fast release of active ingredients (within 30 minutes for both OXP and MPS). Thereafter, further generations were formulated in order to modify the release profiles for both OXP and MPS from a faster release (e.g., within 30 minutes) to a more controlled release (e.g., over a 3- to 4-hour window), without compromising their release efficiencies.
Group 1 (including samples 1-1, 1-2, and 1-3) and Group 1′ (including samples 1′-1, 1′-2, and 1′-3) were loaded with Oxaliplatin 3 mg; mycophenolate sodium 20 mg; Polyacrylic acid-974 0.1 g; DOPA 0.1 g; CMC 0.2 g. Based on the calculations, each formulation (radius=5 mm) of the samples theoretically contained 1.56 μg OXP and 10 μg MPA.
Group 2 (including samples 2-1, 2-2, and 2-3) and Group 2′ (including samples 2′-1, 2′-2, and 2′-3) were loaded with Oxaliplatin 10 mg; mycophenolate sodium 20 mg; Polyacrylic acid-974 0.1 g; DOPA 0.1 g; CMC 0.2 g. Based on the calculations, each formulation (radius=5 mm) of the samples theoretically contained 5.20 μg OXP and 10 μg MPA.
Group 3 (including samples 3-1, 3-2, and 3-3) and Group 3′ (including samples 3′-1, 3′-2, and 3′-3) were loaded with Oxaliplatin 30 mg; mycophenolate sodium 20 mg; Polyacrylic acid-974 0.1 g; DOPA 0.1 g; CMC 0.2 g. Based on the calculations, each formulation (r=5 mm) of the samples theoretically contained 15.6 μg OXP and 10 μg MPA.

The releases of active ingredients in Groups 1, 2, and 3 were performed through a filter, while Groups 1′, 2′ and 3′ were operated directly in a vial.

Sample 1, Sample 2 and Sample 3 were typical pharmaceutical formulations. For Sample 1, each formulation (radius=5 mm) of the samples theoretically contains 1.56 μg OXP and 10 μg MPA. For Sample 2, each formulation (radius=5 mm) of the samples theoretically contains 5.20 μg OXP and 10 μg MPA. For Sample 3, each formulation (radius=5 mm) of the samples theoretically contains 15.6 μg OXP and 10 μg MPA.

Sample A, Sample B, and Sample C were solutions comprising the bioadhesive materials and active ingredients before 3D printing.

Also, a separated sample loaded with OXP and named PAA+OXP, CMC+OXP, and DOPA+OXP (1 cm×1 cm) was prepared. For PAA+OXP, each sample loaded 2.59 mg of OXP. For CMC+OXP, each sample loaded 1.89 mg of OXP. For DOPA+OXP, each sample loaded 0.99 mg of OXP.

### Version 4

Further to the results from the first, second, and third generations as described above, pharmaceutical formulations comprising a multi-layer design were prepared in order to address the undesirable fast- and low-efficiency release of OXP as previously observed. This multi-layer design of the pharmaceutical formulation comprises the same support frame and utilizes two (instead of one) layers comprising the bioadhesive materials and active ingredients.

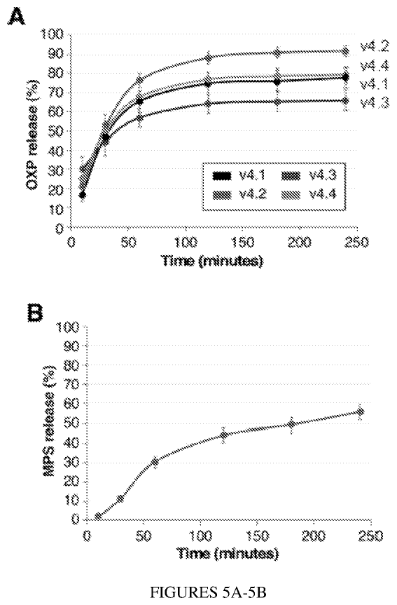

Four variations of the fourth-generation (v4) formulation (i.e., v4.1-v4.4) were fabricated. In vitro active ingredient release studies showed that the v4.2 formulation demonstrated the best release profile of OXP, displaying a sustained release from 0 to 2 hours and reaching a plateau at 91% of the extractable amount after 3 hours (see FIG. 5A). MPS showed almost a linear release profile (zero kinetic) from 0 to 4 hours, reaching a plateau at 55.6% of the extractable amount after 4 hours (FIG. 5B).

### Version 5

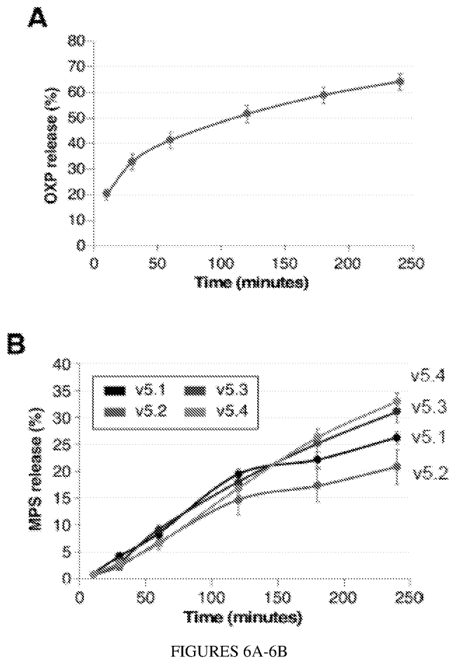

A fifth-generation (v5) pharmaceutical formulation was fabricated based on the v4.2 formulation comprising a fixed amount of OXP but different amounts of MPS. In vitro release studies showed that the release of OXP was controlled and efficient (FIG. 6A). However, the release of MPS, while controlled over the 4-hour window, was not very efficient, falling within the 21-33% range of the total loaded amount of MPS. Relatively speaking, the v5.3 and v5.4 formulations demonstrated the best release profiles of MPS (FIG. 6B).

### Version 6

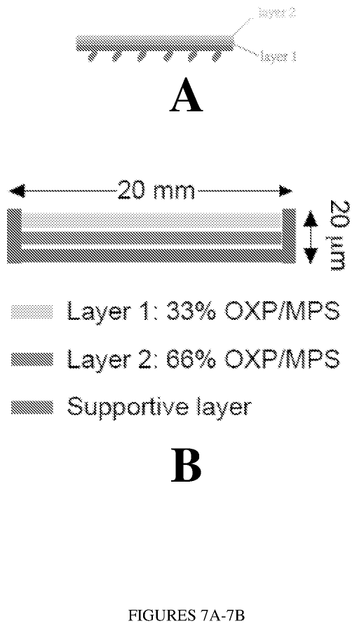

The ratio of OXP and MPS released from the pharmaceutical formulations cumulatively over a 4-hour window is a key indicator of the synergistic effect of these two active ingredients in affecting dysplastic oral keratinocytes. To optimize the pharmaceutical formulations so that OXP and MPS are released in a 1-to-6.4 weight ratio, the sixth-generation (v6) pharmaceutical formulation utilized the v5.3 design with five further variations. The exemplary two-layer designs are shown in FIGS. 7A-7B.

Thus, five variations of the v6 pharmaceutical formulation were tested, each comprising two layers of bioadhesives/active ingredients with the same total amount of MPS (14.13 μg in a 5 mm-diameter circular disc) and with different amounts of OXP. The five pharmaceutical formulations (i.e., v6.1-v6.5) contained a total amount of 7.06 μg, 3.5 μg, 1.77 μg, 1.06 μg, and 7.06 μg OXP, respectively, for a 5 mm-diameter disc. This total amount was distributed in a 1:2 ratio between the top layer:bottom layer and was adjusted to pH 6.0 (for the v6.1-6.4 formulations) or pH 7.4 (for the v6.5 formulation).

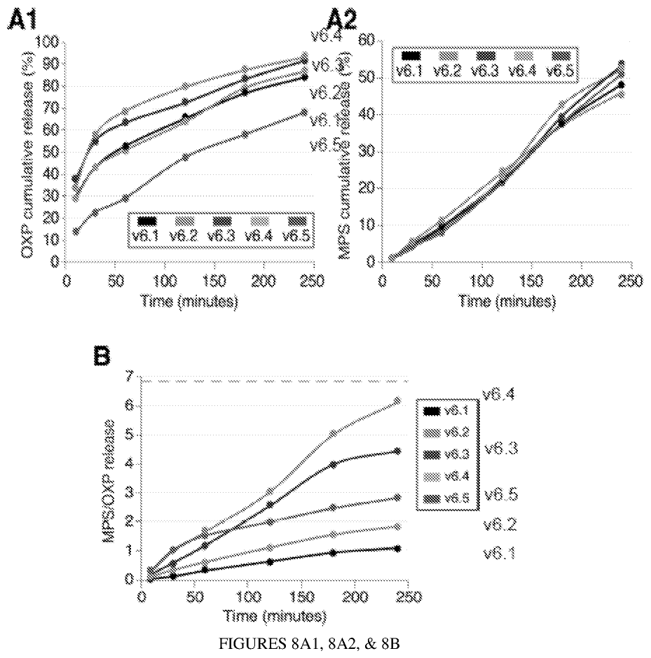

The cumulative release profiles of OXP and MPS at 10 minutes, 30 minutes, 1 hour, 2 hours, 3 hours, and 4 hours were quantified by UPLC-PDA (Ultra-Performance Liquid Chromatography with Photo-Diode-Array detector) and calculated as percentages of their respective total extractable amounts. Within 10 minutes, 30-35% of OXP was released from the v6.1-v6.4 formulations and 13% was released from the v6.5 formulations. All five formulations showed a steady release profile after 30 minutes and reached 68-93% at 4 hours, with the v6.4 formulations demonstrating the highest release efficiency (see FIG. 8A1).

MPS showed a zero-order kinetic release profile from 0-4 hours and a 50% release efficiency at the end of 4 hours (FIG. 8A2). Of the five formulations, only the v6.4 formulations achieved close to the targeted 6.9:1 ratio of the cumulative released MPS:OXP amounts by weight over a 4-hour period (see dashed line of FIG. 8B). The amounts of OXP and MPS released from a 5 mm-diameter circular v6.4 formulation were 855 ng and 5252 ng, respectively, which were about 6 times higher than the estimated amounts required to reach their respective IC₅₀ concentrations in the tissue (128.9 ng and 888.7 ng).
Group 1 (including samples 1-1, 1-2, 1-3, 1-4, 1-5, and 1-6) were made with OXP 10 mg; MPA 20 mg; Polyacrylic acid-974 0.1 g; DOPA 0.1 g; and CMC 0.2 g.

Based on the calculations, each formulation (r=5 mm) of the samples theoretically contains 7.06 μg OXP and 14.13 μg MPA. The content of OXP in the two layers was layer 1:layer 2=2:1, the content of MPA in the two layers was layer 1:layer 2=2:1 at pH 6.
Group 2 (including samples 2-1, 2-2, 2-3, 2-4, 2-5, and 2-6) were made with OXP 5 mg; MPA 20 mg; Polyacrylic acid-974 0.1 g; DOPA 0.1 g; and CMC 0.2 g. Based on the calculations, each formulation (r=5 mm) of the samples theoretically contains 3.53 μg OXP and 14.13 μg MPA. The content of OXP in the two layers was layer 1:layer 2=2:1, the content of MPA in the two layers was layer 1:layer 2=2:1 at pH 6.
Group 3 (including samples 3-1, 3-2, 3-3, 3-4, 3-5, and 3-6) were made with OXP 2.5 mg; MPA 20 mg; Polyacrylic acid-974 0.1 g; DOPA 0.1 g; and CMC 0.2 g. Based on the calculations, each formulation (r=5 mm) of the samples theoretically contains 1.765 μg OXP and 14.13 μg MPA. The content of OXP in the two layers was layer 1:layer 2=2:1, the content of MPA in the two layers was layer 1:layer 2=2:1 at pH 6.
Group 4 (including samples 4-1, 4-2, 4-3, 4-4, 4-5, and 4-6) were made with OXP 1.5 mg; MPA 20 mg; Polyacrylic acid-974 0.1 g; DOPA 0.1 g; and CMC 0.2 g. Based on the calculations, each formulation (r=5 mm) of the samples theoretically contains 1.059 μg OXP and 14.13 μg MPA. The content of OXP in the two layers was layer 1:layer 2=2:1, the content of MPA in the two layers was layer 1:layer 2=2:1 at pH 6.
Group 5 (including samples 5-1, 5-2, 5-3, 5-4, 5-5, and 5-6) were made with OXP 10 mg; MPA 20 mg; Polyacrylic acid-974 0.1 g; DOPA 0.1 g; and CMC 0.2 g. Based on the calculations, each formulation (r=5 mm) of the samples theoretically contains 7.06 μg OXP and 14.13 μg MPA. The content of OXP in the two layers was layer 1:layer 2=2:1, the content of MPA in the two layers was layer 1:layer 2=2:1 at pH 7.4.

The releases of active ingredients in Groups 1, 2, 3, 4, and 5 were performed through a filter, then collected and replaced by fresh water at each time point. In Group 5, the solvent was water (pH 7.4).

Sample 1, Sample 2, Sample 3, Sample 4 and Sample 5 represented the pharmaceutical formulations. The MPA content of layer 1 and layer 2 in all the Samples was 9.42 μg and 4.71 μg. For Sample 1, the OXP content of layer 1 and layer 2 was 4.7 μg and 2.35 μg, respectively (2:1). For Sample 2, the OXP content of layer 1 and layer 2 was 2.35 μg and 1.17 μg, respectively (2:1). For Sample 3, the OXP content of layer 1 and layer 2 was 1.18 μg and 0.59 μg, respectively (2:1). For Sample 4, the OXP content of layer 1 and layer 0.706 was 0.353 μg and 2.35 μg, respectively (2:1). For Sample 5, the OXP content of layer 1 and layer 2 was 4.7 μg and 2.35 μg, respectively (2:1).

### Version 7

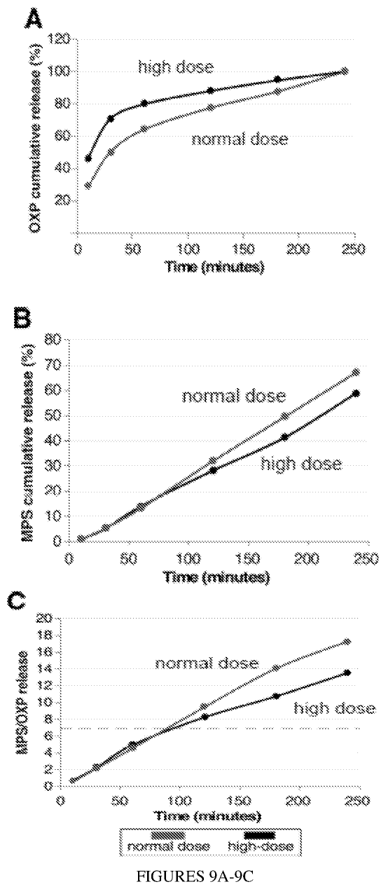

A “high-dose” seventh-generation (v7) pharmaceutical formulation (at 1.5 dose) was also made. Results demonstrated that the v6.4 formulation and the v7 formulation performed equally (see FIGS. 9A-9C).

The v6.4 formulation (low-dose, 6 repeats) was loaded with OXP 1.5 mg; MPA 20 mg; Polyacrylic acid-974 0.1 g; DOPA 0.1 g; and CMC 0.2 g. Based on the calculations, each formulation (r=5 mm) of the samples theoretically contains 1.059 μg OXP and 14.13 μg MPA. The content of OXP in the two layers was layer 1:layer 2=2:1, the content of MPA in the two layers was layer 1:layer 2=2:1.

The v7 formulation (high-dose, 6 repeats) was loaded with OXP 1.5 mg; MPA 20 mg; Polyacrylic acid-974 0.1 g; DOPA 0.1 g; and CMC 0.2 g. Based on the calculations, each formulation (r=5 mm) of the samples theoretically contains 1.5885 μg OXP (1.5×) and 21.195 μg MPA (1.5×). The content of OXP in the two layers was layer 1:layer 2=2:1, the content of MPA in the two layers was layer 1: layer 2=2:1).

### Example 3

### Analysis of In Vivo Mucoadhesiveness of Pharmaceutical Formulations in Rats

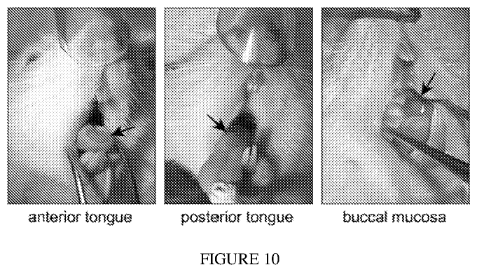

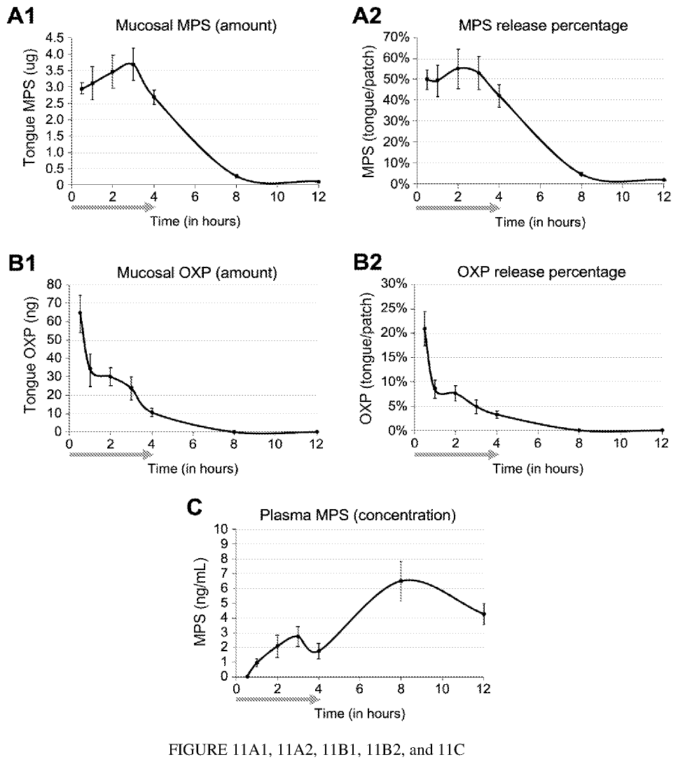

The instant example evaluated the mucosal adhesiveness of the pharmaceutical formulations in Sprague Dawley rats. Two different sizes of the pharmaceutical formulations (4 mm and 3 mm) were analyzed. The pharmaceutical formulations were placed in one of three locations in the oral cavity of the rats: anterior tongue, posterior tongue, or buccal mucoa (see FIG. 10). Each group contained either 8 rats or 10 rats.

Thereafter, for the 4 mm pharmaceutical formulation, it was evaluated if the formulation was still in place at the administered location at time points of 0.5, 1, 2, 1.5, 2, 3, and 4 hours. For the 3 mm pharmaceutical formulation, it was evaluated if the formulation was still in place at the administered location at 4 hours only. The results are shown in Table 1 below and indicate that both pharmaceutical formulations exhibited excellent mucosal adhesiveness.

TABLE 1

| | | Timepoint (hours) | | | | | |
|---|---|---|---|---|---|---|---|
| Diameter | Site | 0.5 | 1 | 1.5 | 2 | 3 | 4 |
| 4 mm | Anterior | 8/8 | 8/8 | 8/8 | 8/8 | 8/8 | 8/8 |
| | tongue | rats | rats | rats | rats | rats | rats |
| | Posterior | 8/8 | 8/8 | 8/8 | 7/8 | 8/8 | 8/8 |
| | tongue | rats | rats | rats | rats | rats | rats |
| | Buccal | 10/10 | 10/10 | 9/10 | 10/10 | 10/10 | 10/10 |
| | | rats | rats | rats | rats | rats | rats |
| 3 mm | Anterior | Not determined | | | | | 8/8 |
| | tongue | | | | | | rats |
| | Posterior | | | | | | 8/8 |
| | tongue | | | | | | rats |
| | Buccal | | | | | | 10/10 |
| | | | | | | | rats |

### Example 4

### Delivery of Mycophenolic Acid and Oxaliplatin from Pharmaceutical Formulation in a Healthy Rat Tongue

The instant example analyzes exemplary pharmaceutical formulations applied to the tongue of healthy rats for different time periods up to 4 hours. Concentrations of mycophenolic acid and oxaliplatin in tongue tissues were measured at the corresponding times and up to 12 hours from the beginning of the formulation application in rats. The following are groups of rats that were studied:
Group #1: Formulation applied for 0.5 hour and rat tongues were harvested for drug analysis. N=8 rats.
Group #2: Formulation applied for 1 hour and rat tongues were harvested for drug analysis. N=13 rats.
Group #3: Formulation applied for 2 hours and rat tongues were harvested for drug analysis. N=13 rats.
Group #4: Formulation applied for 3 hours and rat tongues were harvested for drug analysis. N=12 rats.
Group #5: Formulation applied for 4 hours and rat tongues were harvested for drug analysis. N=6 rats.
Group #6: Formulation applied for 4 hours and removed from the rat. At 8 hours after the start of the Formulation application, rat tongues were harvested for drug analysis. N=7 rats.
Group #7: Formulation applied for 4 hours and removed from the rat. At 12 hours after the start of the Formulation application, rat tongues were harvested for drug analysis. N=9 rats.

Table 2 shows tongue tissue and plasma amounts of MPS and OXP following supra-lingual applications of the pharmaceutical formulation.

TABLE 2
Mean ± SD amount of mycophenolate and oxaliplatin delivered to rat
tongues at various time points following supra-lingual administration up
to 4 hours (each formulation comprised 305 ng of OXP and 5,342 ng of MPSate).

| | Mycophenolate (MPS) | | Oxaliplatin (OXP) | |
|---|---|---|---|---|
| | Tongue Tissue | Plasma | Tongue Tissue | Plasma |
| Time | (ng) | (ng/mL) | (ng) | (ng/mL) |
| Tongue sampled at 0.5 | 2,953 ± 493 | ND^{b} | 65.0 ± 28.7 | ND |
| hour of formulation | | | | |
| application | | | | |
| Tongue sampled at 1 hour | 3,117 ± 1,850 | 0.96 ± 0.26 | 33.7 ± 31.9 | ND |
| of formulation application | | | | |
| Tongue sampled at 2 hours | 3,466 ± 1,825 | 2.09 ± 0.76 | 29.9 ± 17.2 | ND |
| of formulation application | | | | |
| Tongue sampled at 3 hours | 3,692 ± 1,704 | 2.76 ± 0.68 | 23.7 ± 22.6 | ND |
| of formulation application | | | | |
| Tongue sampled at 4 hours | 2,418 ± 883 | 1.76 ± 0.53 | 10.6 ± 5.1 | ND |
| of formulation application | | | | |
| Formulation was applied | 282 ± 126 | 6.50 ± 1.30 | ND | ND |
| for 4 hours to a rat tongue, | | | | |
| then tongue sampled at 4 | | | | |
| hours after the formulation | | | | |
| removal (8 hours from the | | | | |
| beginning of the | | | | |
| formulation application) | | | | |
| Formulation was applied | 109 ± 56 | 4.26 ± 0.71 | ND | ND |
| for 4 hours to a rat tongue, | | | | |
| then tongue sampled at 8 | | | | |
| hours after the formulation | | | | |
| removal (12 hours from | | | | |
| the beginning of the | | | | |
| formulation application) | | | | |

MPS was delivered rapidly to the tongue tissue. For example, a mean of 2,953 ng of MPS (about 55% of the formulation drug load) was detected from the rat tongue at 0.5 hour supra-lingual formulation application. When a formulation was applied longer via supra-lingue such as 1, 2, 3 or 4 hours, the mean amount of the active ingredient detected in tongue tissue at these corresponding time points were increased slightly and reached the maximum of 3,692 ng in rat tongues of 3-hour formulation application group. A maximum formulation application duration of 4 hours was investigated and found that mean MPSe amount tongue tissues at the end of the 4-hour formulation application was 2,418 ng. Corresponding plasma MPS concentrations were less than 10 ng/mL in all groups. This was consistent with previous pharmacokinetic study results.

Oxaliplatin was delivered to rat tongue tissue rapidly following supra-lingual formulation application. For example, maximum oxaliplatin amount of 65 ng (about 21% of the formulation active ingredient load) was found in the 0.5-hour formulation application group. The amount was gradually decreased when formulation application time increased and no oxaliplatin was detectable after formulation removal at 4 hours. Also consistent with the pharmacokinetic studies, there was no detectable oxaliplatin in plasma in any groups.

FIGS. 11A-11C show the in vivo release profiles of OXP and MPS from the pharmaceutical formulation. As shown, the pharmaceutical formulation effectively delivered MPS and OXP to the target tongue tissue with the selected application time of 0.5, 1, 2, 3 or 4 hours via supra-lingual administration in rats. The active ingredient levels in the tongue tissue were significant and maintained at least during the formulation application time period. Negligent plasma levels of both active ingredients were measured. The data demonstrated excellent delivery characteristics of the pharmaceutical formulation for potential targeted cancer therapy.

### Example 5

### Delivery of Mycophenolic Acid and Oxaliplatin from Pharmaceutical Formulation in a Diseased Rat Tongue

The instant example analyzes exemplary pharmaceutical formulations applied to the tongue of diseased rats. Rats were first treated with 4-Nitroquinoline 1-oxide (4NQO) and monitored until they developed oral cancer. Then each rat was treated with a 4 mm pharmaceutical formulation comprising MPS and OXP via supra-lingual administration for 2 hours daily for a total of 5 consecutive days. Plasma samples were collected from the rats immediately before the application of the fifth dose. Pharmacodynamics were characterized in the rats at the end of treatment regimen.

Table 3 shows plasma concentrations of active ingredients and pharmacodynamic characteristics of the treated rats. Steady-state trough MPS concentrations in the plasma ranged from 2.37 ng/ml to 8.24 ng/mL. These results were consistent from single dose pharmacokinetic studies obtained with the pharmaceutical formulations. The pharmaceutical formulations showed consistent pharmacokinetics following multiple-dose to steady-state dose regimen with significantly improved cancer inhibition in the rat tumor model.

TABLE 3
Plasma concentrations of mycophenolic acid in 4NQO induced
rats following daily 2-hours supra-lingual administration
of the pharmaceutical formulation (each formulation
comprised 9,040 ng MPS and 680 ng OXP)

| | Plasma concentration | Plasma concentration | |
|---|---|---|---|
| | (ng/mL) of mycophenolate | (ng/ml) of oxaliplatin | |
| | immediately before the | immediately before the | Pharmaco- |
| Rat ID | fifth dose application | fifth dose application | dynamics |
| 14-12-1 | 8.24 | Not detectible (ND) | G0 |
| 13-10-4 | 6.84 | ND | G0 |
| 15-3-2 | 3.83 | ND | G0 |
| 15-2-5 | 2.54 | ND | G0 |
| 17-5-2 | 4.82 | ND | G0 |
| 17-1-3 | 2.37 | ND | G0 |
| 23-3-4 | 5.18 | ND | G0 |

No detectable levels of oxaliplatin in plasma was observed. This was also consistent with the single dose formulation pharmacokinetic studies. Pharmacodynamic data showed significant improvement of cancer tumor inhibition.

### Example 6

### Design and Analysis of Pharmaceutical Formulations for Oral Dysplasia

The instant example provides analysis of pharmaceutical formulations on oral dysplasia in rats.

### Materials and Methods

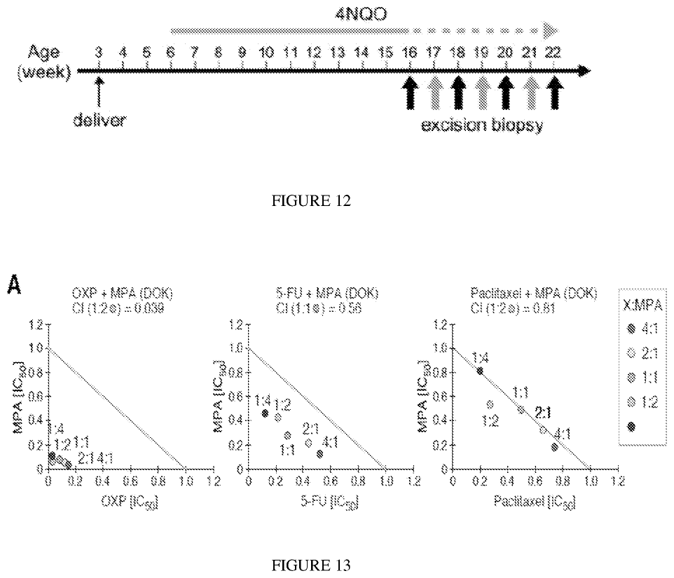

1. Animal care and treatment. Animals were housed by the Program for Animal Resources at the TAMHSC-Houston campus and handled in accordance with the principles described by the Guide for the Care and Use of Laboratory Animals and the procedures approved by the IACUC (2017-0287-IBT and 2020-0232-IBT). Male Sprague-Dawley (SD) rats (3-week-old) were purchased from ENVIGO (Indianapolis, IN), housed in a room with a barrier system, and maintained at 23±2° C., 55±5% relative humidity, and a 12-hour light-dark cycle, with free access to chow and drinking water. After 3 weeks of acclimatization, rats were fed with 4NQO water for OPL induction. 4NQO (N0250, TCI America) was dissolved in propylene glycol to make a 4 mg/mL stock solution and diluted to 50 μg/mL (50 ppm) in acidified distilled water. Throughout the feeding process (10-16 weeks), rats were monitored for their body weight, food/water intake, and motor activity. 4NQO water was replaced twice a week. After 8-10 weeks of 4NQO feeding, incisional biopsies were taken from mucosal lesions on the dorsal tongue surface for histopathological diagnosis every other week (FIG. 12). Rats with histologically confirmed OEDs were withdrawn from 4NQO feeding and received formulation treatment. The remaining lesions were treated for 2 hours a day for 5 consecutive days. Punch biopsies were taken to determine the efficacy of formulation treatment 10 days after the last dose of formulation treatment. Lesions were graded as 0, 1, 2, or 3 for no dysplasia, low-grade dysplasia (≤½ epithelial thickness), high-grade dysplasia (>½ epithelial thickness), or invasive carcinoma, respectively.
2. Combination index (CI) and isoborogram. Dysplastic oral keratinocyte (DOK) cells were purchased from Sigma-Aldrich (Cat #94122104) with Certificate of DNA Profile Analysis and grown in DMEM supplemented with 10% FBS, 2 mM glutamine, and 5 μg/ml hydrocortisone. Tumor growth-inhibitory curves based on the MTT assay were plotted over a range of five active ingredient concentrations with three-fold increment. The half-inhibitory concentrations (IC₅₀) were calculated using the Calcusyn program (Biosoft, Ferguson, MO). Drug interaction effects were measured by mixing two active ingredients at a 4-to-1, 2-to-1, 1-to-1, 1-to-2, or 1-to-4 ratio of their respective IC₅₀ concentrations. Each combination was tested for its tumor growth-inhibitory effect over a range of five different dosages. CI values were calculated from 3-4 independent experiments using the Calcusyn program.
3. Pharmaceutical formulation. Formulations were designed to contain multiple layers of bioadhesive materials and active ingredients on a supportive frame (Tegaderm™ transparent film). The supportive frame prevents water leakage without affecting oxygen permeation. Fabrication was carried out using a CELLINK_BIO_X_3 D printer. Bioadhesive materials include polyacrylic acid (PAA, Carbopol® 974 polymer), gelatin-modified dopamine (DOPA), and carboxymethyl cellulose (CMC). To fabricate a single-layered OXP-MPS formulation, bioadhesive materials (e.g., 0.1 gm of polyacrylic acid-974 (PAA-974), 0.1 gm of DOPA, and 0.2 gm of CMC) were combined with 1.5 mg of OXP (102544-196, Sigma) and 20 mg of MPS (1448989, Sigma), dissolved in 10 ml distilled water, and blended in a Thinky Mixer (ARE-310, THINKY USA Inc.) for 40 minutes to form a homogenous aqueous solution. The homogenous aqueous solution was added to a syringe with a 300 μm diameter nozzle and printed in a square sheet (30 mm×30 mm×20 μm, containing 48.56 μg of OXP and 648 μg of MPS) directly on the supportive frame. During the printing process, the flow rate was controlled by adjusting the dispensing pressure, the valve opening time, and the dosing distance. The width of the printed lines was controlled by adjusting the printing speed and the parameters mentioned above. To fabricate a double-layered OXP-MPS v6.4 formulation (30 mm×30 mm×20 μm) with a top-to-bottom active ingredient ratio of 1-to-2 (FIG. 7B), bioadhesive materials were prepared as described above, evenly divided into two parts, and mixed with OXP and MPS in the amounts of 0.5 mg/6.67 mg (OXP/MPS) for the top layer and 1.0 mg/13.33 mg for the bottom layer. Square sheets (30 mm×30 mm) were printed directly onto the supportive frame layer by layer (FIG. 1B), with a 24-hour drying time at 37° C. after each layer was printed, reducing the thickness down to 20 μm. For testing and application, circular formulations of different diameters were punched using a Miltex biopsy punch with plunger (4MD Medical, Lakewood, New Jersey).
4. UPLC-PDA (Ultra-Performance Liquid Chromatography with Photo-Diode-Array detector). UPLC-PDA was used to measure the amounts of OXP and MPS in the formulations. 5 mm-diameter circular discs of formulations were sonicated in 1 mL H₂O for 20 minutes in an ice-water bath and vortexed. 45 μL of supernatant was mixed with 5 μL of 10× working buffer, 10 μL of internal standard (AMP 50 μg/mL, WF 100 μg/mL), and 200 μL of acetonitrile/0.01% ammonium hydroxide. 10 μL of the mixture was injected into UPLC and detected by PDA. Assays were linear from 0.1 μg/mL to 10.0 μg/mL for OXP and from 0.1 μg/mL to 50.0 μg/mL for MPS. The accuracy and precision were within acceptable criteria of 10% for both assays.
5. LC-MS/MS. The amounts of OXP and MPS in the plasma were measured by the LC-MS/MS methods. The amounts of OXP and MPS in the plasma were measured using LC-MS/MS with a Shimadzu Nexera X2 UPLC (Columbia, MD) and a 4000 QRRAP® MS/MS system (AB Sciex, Redwood City, CA). System control and data analysis were performed using Analyst® software 1.6.2 (Sciex, Redwood City, CA). To measure the amount of OXP in the plasma, 20 μL of sample was mixed with 4 μL of internal standard (antipyrine 100 ng/mL) and 80 μL of acetonitrile/0.01% ammonium hydroxide, and 10 μL of the mixture was injected into LC-MS/MS. Chromatographic separation of MPS was performed on an ACE Excel 2 Super Cis column (50×2.1 mm, 2 μm, UK) with a binary solvent system of 0.1% formic acid in water (solvent A) and 0.1% formic acid in acetonitrile (solvent B). Chromatographic separation of OXP was performed on a Phenomenex Lux 5u Cellulose-1 column (250×4.6 mm, 5 μm) with an isocratic elution of 50% acetonitrile in water containing 1.25 mM ammonia formate. The injection volume was 5 μL, the total run time was 5.5 minutes, and the flow rate was 0.8 mL/min. Tandem mass spectrometry was employed under positive ESI to detect the specific precursor to product ion transitions m/z 398.1→306.0 for oxaliplatin and m/z 189.0→131.0 for IS. Source parameters including ion spray voltage, temperature, nebulizer gas and heater gas pressure were set at 5000V, 700° C., 60 and 55 psi, respectively. The linear response ranged from 10 ng/ml to 2500 ng/ml (r²>0.990). The LLOQ for OXP and MPS by LC-MS/MS methods is 10 ng/mL and 0.5 ng/mL, respectively.
6. In vitro active ingredient release test. In vitro active ingredient release profiles were tested in aqueous solution, where individual OXP-MPS formulations in 5 mm-diameter circular discs were immersed in 1.5 mL distilled water in a Slide-A-Lyzer™ MINI dialysis device at 37° C. At designated time points (10, 30, 60, 120, 180, and 240 minutes), 1 mL of the solution was collected from the device and replenished with the same amount of fresh distilled water. Collected solution was filtered through 0.45 μm nylon filters and quantified for the amounts of OXP and MPS by UPLC-PDA. Six samples were repeated for each timepoint. The amounts of cumulative release were reported as mean±standard deviation.
7. Side leakage test. Active ingredient leakage from the side of the v6.4 formulations was measured in the Franz Diffusion Cell Apparatus. A 9 mm-diameter circular disc of formulation was adhered to the undersurface of the porous membrane and immersed in the bottom receptor chamber, containing 5 mL of water and maintained at 37° C. The donor chamber was filled with water. Aliquots of solution (1 mL) from the receptor chamber were sampled for active ingredient measurement at different timepoints (10 minutes, 30 minutes, 1 hour, 2 hours, and 4 hours), and calculated as percentages of the extractable active ingredient amounts from the formulations.
8. Ex vivo mucosal permeation test. Porcine buccal tissue was purchased from a local slaughterhouse (J&J Packing Company, Inc). The buccal mucosa was prepared by trimming the buccal tissue down to a 5-7 mm thickness and mounted between the donor and receptor chambers of a Franz Diffusion Cell Apparatus. A v6.4 formulation (d=9 mm) was then attached to the mounted buccal mucosa, with its bioadhensive layer facing down in the donor chamber. Both the donor and receptor chambers were filled with water and maintained at 37° C. The receptor chamber solution was stirred at 400 rpm. The amounts of OXP and MPS permeating through the buccal mucosa into the receptor chamber were measured by LC-MS/MS at 10 minutes, 30 minutes, 1 hour, 2 hours, and 4 hours. The amounts of active ingredients remaining in the buccal mucosa and the formulation after 4 hours were measured by LC-MS/MS and UPLC-PDA, respectively.
9. Mechanical property and bioadhesive strength tests. The mechanical strength of the v6.4 formulations was measured by a texture analyzer (SMT1-22, TestResources Inc.). Tested samples had a width of 20 mm, a gauge length of 6 cm and a thickness of 0.4 mm. The length between the clamps was set to 20 mm and the speed of testing was set to 5 mm/min. From machine-recorded data, the stress-strain relationship was calculated based on the following equations: (1) stress (σ)=F/A, where F is the applied force and A is the cross-sectional area, and (2) strain (ε)=ΔL/L, where ΔL is the change in length and L is the length between the clamps. Elastic modulus was calculated by the slope of the linear regression line best fitting for the initial linear part of the stress-strain curve. Adhesion force was measured using the same texture analyzer. A piece of porcine mucosa was fixed to the base of the test machine, and a formulation was attached to the upper probe of the test machine through a clamp. Adhesion was done by adding 10 μL water to the mucosal surface and immediately lowering the probe to allow contact between the formulation and the mucosa. After applying a 5N force for 30 s, the probe was lifted upwards at a speed of 5 mm/min. Adhesion force (in newton, N) was recorded as the force required to detach the formulation from the mucosal surface.
10. Ex vivo mucoadhesion test. Mucosal tissues were harvested from pig buccal membrane and fixed on glass slides with glue. Formulations were wetted in simulated saliva for seconds, mounted on the porcine mucosa with light pressure, held for 30 seconds, and submerged in simulated saliva at 37° C. A total of 12 formulations were mounted. The number of formulations remaining on the mucosa surface were counted at the indicated time points (0 h, 1 h, 1.5 h, 2 h, 2.5 h, 3.5 h, and 4 h).
11. In vivo mucoadhesion and active ingredient release tests. SD rats (6-8 weeks old) were anesthetized by isoflurane using an induction chamber first and then a custom-made 8-way distributor manifold connected to nasal masks with individual ball valves for separate control over the isoflurane flow rate for maintenance. Circular discs of 3 mm or 4 mm diameter were punched from the mucoadhesive formulations using a Miltex biopsy punch, moisturized with 1-2 μl sterile water, and placed on the dorsal tongue or buccal mucosa with light pressure. For in vivo mucoadhesion tests, rats were given formulations of 3 mm or 4 mm diameter on different locations and examined at different time points (0.5, 1, 1.5, 2, 3, or 4 hours) to determine whether the formulation detached. Eight to ten rats were independently tested for every formulation size, location, and timepoint. For in vivo active ingredient release tests, SD rats received a 4-mm formulation on the dorsal tongue surface and sacrificed at fixed timepoints (0.5, 1, 2, 3, or 4 hours) to collect tongue tissue samples underneath or surrounding the formulation and plasma samples for LC-MS/MS analyses. For two additional sets of rats, formulations were applied on the dorsal tongue surface for 4 hours and removed. Tongue tissues and plasma samples were collected at 4 and 8 hours after formulation removal.
12. Histopathological analysis. Tongue lesions were collected by incisional or punch biopsy, fixed in 10% buffered formalin, embedded in paraffin blocks, and sectioned for H&E staining. Sections were coded in a double-blind manner and examined by a board-certified oral pathologist. Hyperplasia is defined as thickening (increased cell number) of the spinous cell layer of the surface epithelium without cellular atypia. Dysplasia is defined by the presence of atypical cellular and/or architectural changes, indicating abnormal maturation. Those changes include hyperchromatism, pleomorphism, increased nuclear-to-cytoplasmic ratio, rounding of the rete ridges, nuclear crowding, multi-nucleation, and dyskeratosis. Low-grade and high-grade dysplasia are defined by cellular and/or architectural atypia present within or beyond the lower half of the epithelium, respectively. Oral squamous cell carcinoma is defined as a malignancy arising from the surface squamous epithelium that has broken through the basement membrane and invaded the underlying tissue, such as the skeletal muscle.

### Mycophenolic Acid (MPA) Synergizes the Cytotoxicity of Oxaliplatin (OXP) in Dysplastic Oral Keratinocyte (DOK) Cells

The instant example evaluated the chemo-synergizing effect of MPA in combination with 5-FU, paclitaxel, or OXP in DOK cells. The IC₅₀ concentrations of MPA (13.30 μM), OXP (1.67 μM), 5-FU (3.33 μg/mL), and paclitaxel (0.41 nM) in DOK cells were determined by their dose response curves based on the MTT assay. The interactions between MPA and one of 5-FU, paclitaxel, or OXP were analyzed by an Isoborogram. Synergistic (strong, moderate, and mild) and additive drug interactions were defined by their CI value range of 0.1-0.3, 0.3-0.7, 0.7-0.85, and 0.85-1.0, respectively. Five different ratios of the active ingredients (4:1, 2:1, 1:1, 1:2, and 1:4) were tested for each combination, with the 1:1 ratio representing each active ingredient at equal amounts with respect to their IC₅₀ concentrations. In DOK cells, MPA showed strong cytotoxic synergy with OXP at all five ratios tested but only mild-to-moderate synergy with 5-FU (CI=0.56) or paclitaxel (CI=0.81) (FIG. 13). Based on the synergy data, a mucoadhesive pharmaceutical formulation was designed to topically deliver a combination of OXP and MPA. The v6.4 pharmaceutical formulation described in Example 2 was utilized for the instant example.

### In Vitro Properties of the OXP-MPS v6.4 Pharmaceutical Formulation

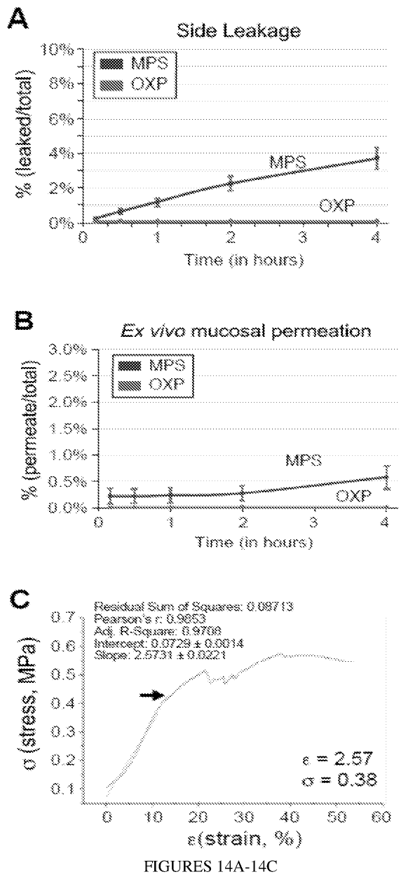

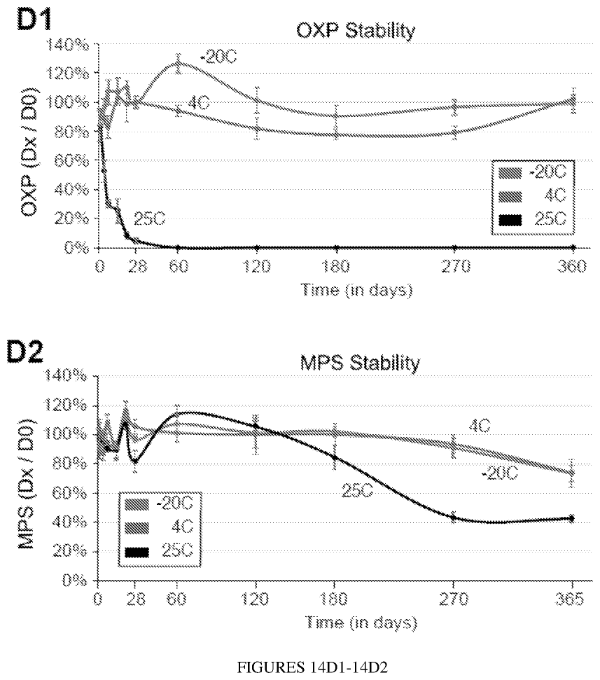

Leakage from the side of the v6.4 formulation was measured in a Franz Diffusion Cell apparatus and calculated as a percentage of the total amount embedded in the formulation. Only a trace amount of MPS (3.7%+0.61%, SEM) leaked from the side of the formulation during a 4-hour period and no OXP was detected (FIG. 14A).

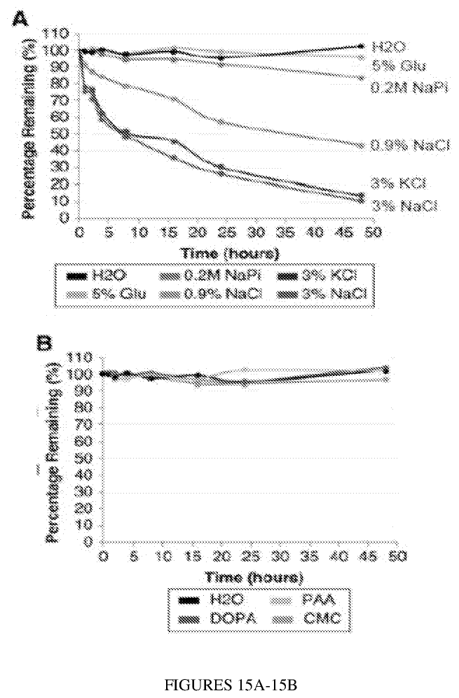

Ex vivo mucosal permeation of OXP and MPS was measured on a piece of porcine mucosa (5-7 mm thickness). At the end of 4 hours, 45.1% (+7.7%) of the MPS was released and retained in the mucosa, and only a very small fraction (0.5%) of the MPS passed through the mucosal/submucosal tissue to reach the receptor chamber (FIG. 14B). No OXP was detected in the receptor chamber or in the ex vivo mucosa throughout the 4-hour duration. It has been reported that OXP undergoes rapid conversion to Pt(dach)Cl(OH) (monochloro DACH platin) and Pt(dach)Cl₂ (dichloro DACH platin) in the presence of chloride ion. These chloride derivatives of OXP are not detectable by current available assays. In support, it was determined that OXP is converted by chloride in a time and concentration-dependent manner but remains stable in NaPi, glucose, or H₂O solutions, as well as in solutions containing the bioadhesive material components (FIGS. 15A and 15B). It is worth noting that even though these chloride derivatives of OXP are not detectable, they are known to be as cytotoxic as OXP in cellular assays.

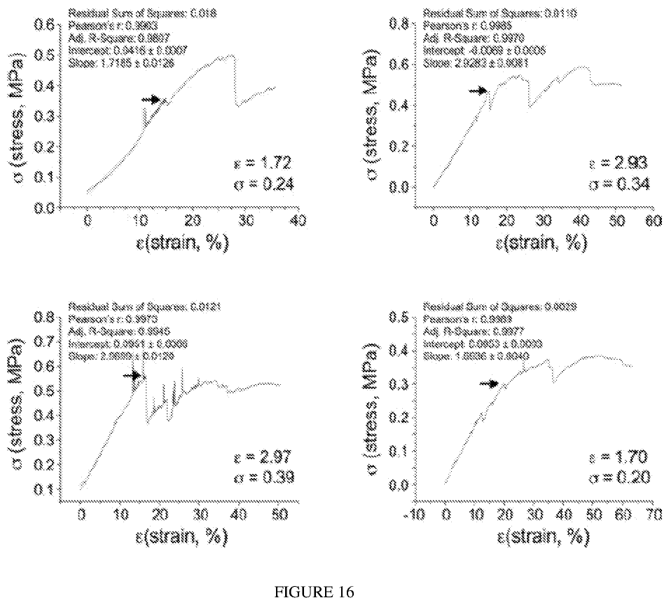

The average elastic modulus of five independent v6.4 formulations was determined to be 2.38 MPa (+0.63 SD) using a TestResources System (FIG. 14C, FIG. 16). The elasticity of the v6.4 formulation was shown by the stress-strain curves that it could be stretched for over 30% longer than its original length without breaking (arrows). Adhesion force is widely used to characterize the bioadhesive strength of a mucoadhesive formulation. It is defined by the force required to detach the formulation from the mucosal surface that it contacts.

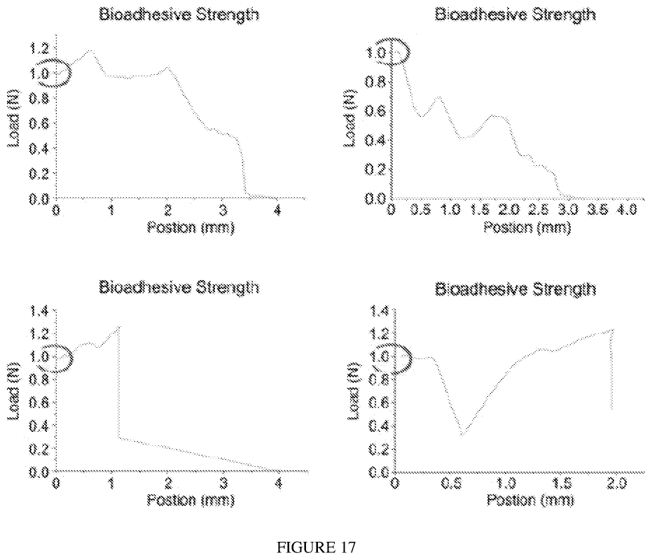

The average adhesion force of the OXP-MPS v6.4 formulation mounted on porcine buccal mucosa was 1.29±0.30 N (mean±s.d.) (FIG. 17). This finding indicates that the formulation can be readily peeled off after active ingredient release. To determine active ingredient stability, v6.4 formulations were made and stored at different temperatures (25° C., 4° C., or −20° C.) for 1 day, 4 days, 7 days, 14 days, 21 days, 28 days, 2 months, 4 months, 6 months, 9 months, and 12 months. OXP and MPS remaining in stored formulations were extracted, quantified by UPLC-PDA, and compared to the amounts extracted from freshly made formulations (0 days).

OXP in the v6.4 formulation remained stable for at least 12 months if stored at 4° C. or −20° C., but becomes rapidly degraded in 4 days when kept at 25° C. (FIG. 14D1). MPS remains stable for 6 months even at 25° C. If stored at −20° C. and 4° C., MPS is stable for 12 months (FIG. 14D2).

### Ex Vivo and In Vivo Mucoadhesiveness Properties of OXP-MPS v6.4 Formulation

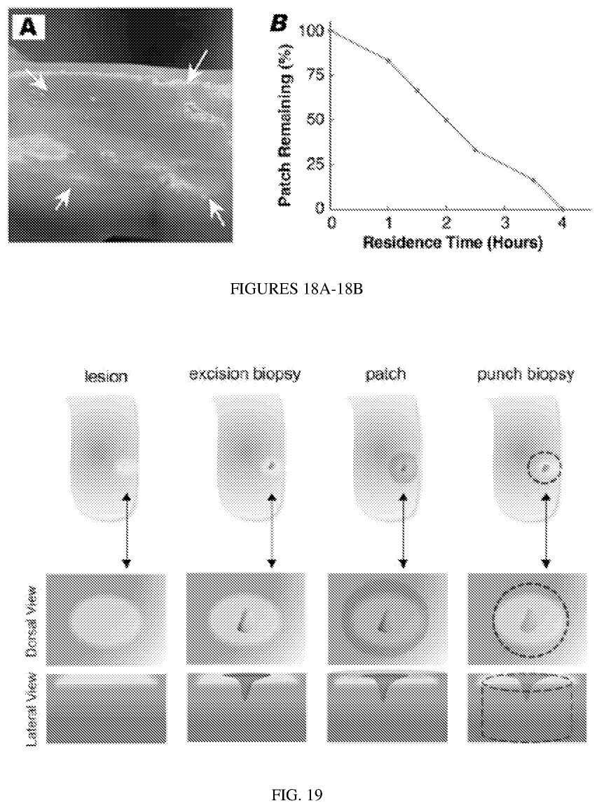

Ex vivo mucoadhesiveness of the OXP-MPS v6.4 pharmaceutical formulations was tested on a piece of porcine buccal mucosa completely submerged in simulated saliva (see FIG. 18A). About 50% of the formulations stayed on the mucosa after 2 hours and >80% of the formulations detached from the mucosa after 3.5 hours (see FIG. 18B). The average residence time was 2.4 hours.

The in vivo mucoadhesiveness of the OXP-MPS v6.4 formulations on oral mucosal surface was determined at different sites. Circular discs of the formulations (4 mm or 3 mm diameter) were placed on the anterior dorsal tongue, posterior dorsal tongue, and buccal mucosa (FIG. 10). Retention was examined at fixed timepoints while animals were kept under anesthesia. Results showed that almost all formulations stay on the mucosa for at least 4 hours, regardless of their size or the placement site (see Table 1).

### In Vivo Active Ingredient Release Profiles

The release profiles of OXP and MPS from the v6.4 pharmaceutical formulation was evaluated and results were described herein in Example 4.

### Efficacy of the OXP-MPS v6.4 Formulation in Treating OEDs in a 4NQO Rat Model

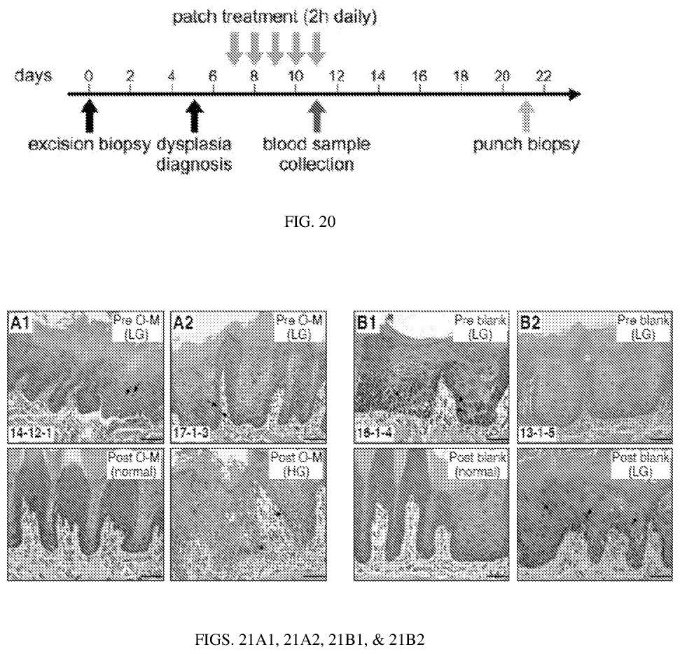

The efficacy of the OXP-MPS v6.4 formulations in treating OEDs in vivo was tested using a 4NQO Sprague Dawley (SD) rat model (FIG. 12). Animals with pathologically confirmed low-grade OEDs were randomly divided to receive the OXP-MPS (O-M) formulation or non-medicated control formulation, collected for histological evaluation after a 5-day treatment course and a 10-day recovery period (FIG. 19 and FIG. 20).

As shown in FIG. 21A1, FIG. 21B1 and Table 4, 88.9% of low-grade OEDs treated with the OXP-MPS formulations showed a complete reversion to normal epithelium with normal cellular maturation, whereas only 20% of those treated with the control reverted to normal epithelium. Only 11.1% of low-grade OEDs treated with the OXP-MPS formulations remained as dysplastic and 80% of those treated with the control either remained as low-grade OEDs or advanced to invasive OSCC (see FIG. 21A2, FIG. 21B2 and Table 4). Chi-square analysis confirmed that the effect of the OXP-MPS formulations in treating low-grade OEDs was significantly better than that of the controls (p=0.0027) (Table 5).

TABLE 4
Numbers of cases with different histopathological outcomes after 10 d of recovery
in the OXP-MPS (O-M) formulation or control (blank) treatment group.

| | | | | Post-formulation biopsy (10 days) | | |
|---|---|---|---|---|---|---|
| | | Pre-formulation biopsy | | Histopathology | | |
| Formulation | Treatment | Histopathology | Depth | (Grade) | Depth | No. |
| OXP-MPS | 2 hours × | Low-grade | ≤½ | Normal | 0 | 0 |
| (O-M) | 5 days | dysplasia | | epithelium (G0) | | |
| | 2 hours × | Low-grade | ≤½ | Normal | 0 | 1 |
| | 5 days | dysplasia | | maturation with | | |
| | | | | hyperkeratosis (G0) | | |
| | 2 hours × | Low-grade | ≤½ | Normal maturation | 0 | 7 |
| | 5 days | dysplasia | | with basilar | | |
| | | | | hyperplasia (G0) | | |
| | 2 hours × | Low-grade | ≤½ | Low-grade | ≤½ | 0 |
| | 5 days | dysplasia | | dysplasia (G1) | | |
| | 2 hours × | Low-grade | ≤½ | High-grade | >½ | 1 |
| | 5 days | dysplasia | | dysplasia (G2) | | |
| | 2 hours × | Low-grade | ≤½ | SCC (G3) | | 0 |
| | 5 days | dysplasia | | | | |
| Control | 2 hours × | Low-grade | ≤½ | Normal | 0 | 0 |
| (blank) | 5 days | dysplasia | | epithelium (G0) | | |
| | 2 hours × | Low-grade | ≤½ | Normal | 0 | 0 |
| | 5 days | dysplasia | | maturation with | | |
| | | | | hyperkeratosis (G0) | | |
| | 2 hours × | Low-grade | ≤½ | Normal maturation | 0 | 2 |
| | 5 days | dysplasia | | with basilar | | |
| | | | | hyperplasia (G0) | | |
| | 2 hours × | Low-grade | ≤½ | Low-grade | ≤½ | 5 |
| | 5 days | dysplasia | | dysplasia (G1) | | |
| | 2 hours × | Low-grade | ≤½ | High-grade | >½ | 0 |
| | 5 days | dysplasia | | dysplasia (G2) | | |
| | 2 hours × | Low-grade | ≤½ | SCC (G3) | | 3 |
| | 5 days | dysplasia | | | | |

TABLE 5
Chi-square analyses of the efficacy of the OXP-MPS (O-M)
formulation (n = 9) vs. the control (blank) (n =
10) in reversing low-grade OEDs to normal epithelium (p = 0.0027).

| | Outcome (grade) | | |
|---|---|---|---|
| | G0 | ≤G1 | Total |
| OXP-MPS (O-M) | 8 | 1 | 9 |
| Formulation | | | |
| Control (blank) | 2 | 8 | 10 |
| Total | 10 | 9 | 19 |

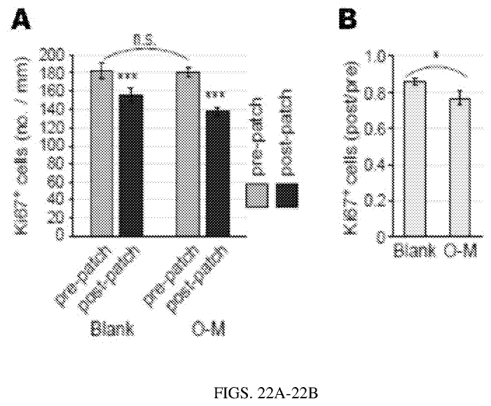

The plasma concentrations of active ingredients, sampled right before the placement of the last (fifth) formulation, were not detectible for OXP and 4.52 (+2.23) ng/ml for MPS in the group treated with OXP-MPS formulations. Ki67 staining showed that the numbers of mitotic cells in the basal and parabasal epithelium were indistinguishable between the OXP-MPS formulation and control groups before the treatment (see FIG. 22A, wherein “patch” refers to the formulation). After the treatment, the numbers of mitotic cells decreased in both groups (see FIG. 22A, wherein “patch” refers to the formulation). By comparison, the OXP-MPS formulation-treated samples demonstrated a higher decrease in their mitotic activity than did the control-treated samples (see FIG. 22B). These results demonstrate the efficacy of the OXP-MPS v6.4 formulations in ablating low-grade OEDs in vivo and reducing the epithelial hyperactive proliferation.

### Local and Systemic Toxicities of the OXP-MPS v6.4 Formulation in Rats

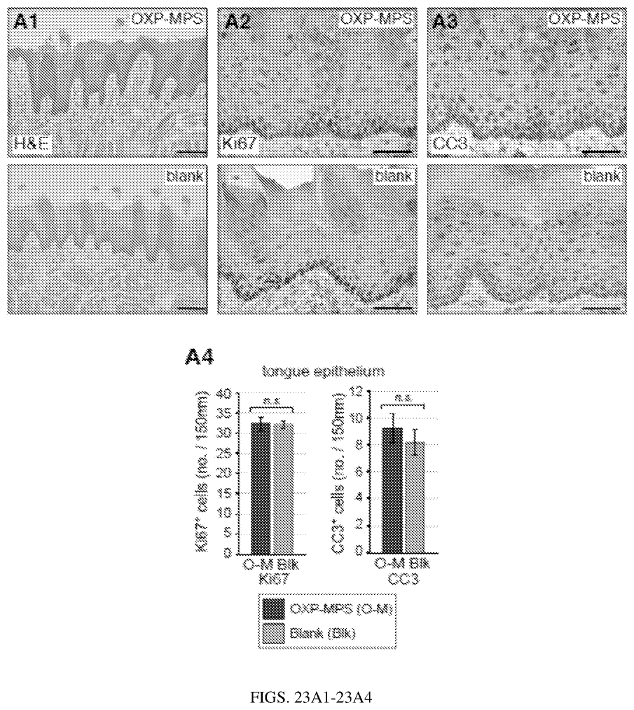

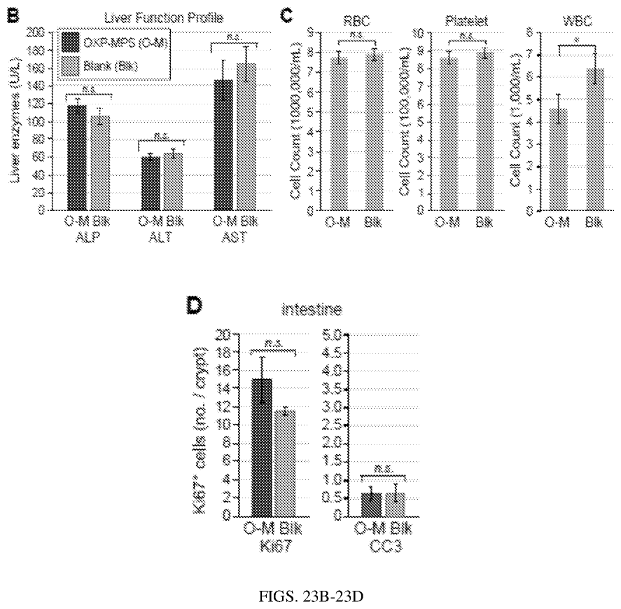

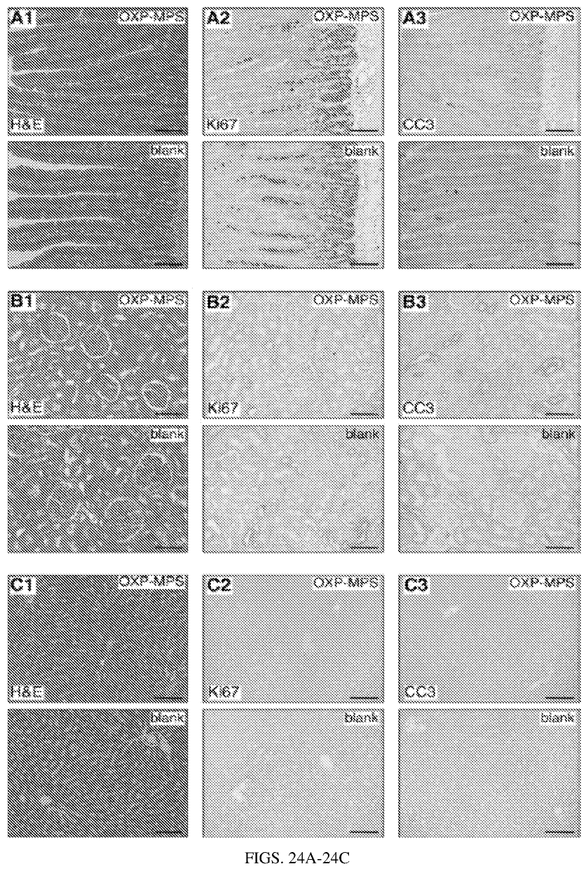

Local and systemic toxicities associated with treatment using the v6.4 formulations were assessed by placing a 4 mm-diameter formulation on the dorsal tongue surface of healthy Sprague Dawley (SD) rats for 4 hours per day for 5 consecutive days. Tissues (tongue, kidney, liver, and intestine) were collected for histological analysis and blood samples for liver function tests and complete blood count (CBC) two days after the last formulation placement. H&E staining showed that the OXP-MPS formulation did not damage the local oral epithelial morphology compared to control (FIG. 23A1). Immunostaining of Ki67 for proliferative cells and cleaved caspase 3 (CC3) for apoptotic cells showed that the OXP-MPS formulation treatment did not affect the normal proliferative activity of basal cells, nor did it increase the number of apoptotic cells after 2 days of recovery (FIGS. 23A2, 23A3, and 23A4). Two tailed t-tests confirmed that there was no difference in the levels of liver enzymes (ΔLP, ΔLT, and AST) (FIG. 23B) or the numbers of RBCs and platelets in the peripheral blood between rats treated with the OXP-MPS formulation vs. control (FIG. 23C). A mild decrease in WBCs was noted in the OXP-MPS formulation-treated group compared to the control group (FIG. 23C). No difference in histopathology (H&E), cell proliferation (Ki67), and cell death (CC3) was found in the intestine, kidney, or liver between rats treated with the OXP-MPS formulation or the control (FIG. 23D and FIG. 24). These results demonstrate that the OXP-MPS formulation does not cause any discernible local or systemic toxicity except for mild leukopenia.

1. A pharmaceutical formulation comprising
i) a support frame,
ii) one or more bioadhesive materials,
iii) a first active ingredient, and
iv) a second active ingredient.

2. The pharmaceutical formulation of claim 1, wherein the pharmaceutical formulation is configured as a three-dimensional printed composition.

3. (canceled)

4. (canceled)

5. The pharmaceutical formulation of claim 1, wherein the one or more bioadhesive materials are selected from the group consisting of polyacrylic acid (PAA), gelatin-modified dopamine (DOPA), carboxymethyl cellulose (CMC), polylactic acid, sodium carboxymethyl cellulose, carbopol, chitosan, PEG (Polyethylene glycol), sodium alginate, gelatin, pectin, Poly(vinyl alcohol), Poly(ethylene oxide), Poly(vinyl pyrrolidone), methylcellulose, methylethyl cellulose, gum tragacanth, soluble starch, and any combination thereof.

6. The pharmaceutical formulation of claim 1, wherein the one or more bioadhesive materials are selected from the group consisting of polyacrylic acid (PAA), gelatin-modified dopamine (DOPA), carboxymethyl cellulose (CMC), and any combination thereof.

7. The pharmaceutical formulation of claim 1, wherein the first active ingredient is oxaliplatin (OXP).

8. The pharmaceutical formulation of claim 1, wherein the second active ingredient is mycophenolic acid (MPA).

9. The pharmaceutical formulation of claim 1, wherein the second active ingredient is mycophenolate (MPS).

10. The pharmaceutical formulation of claim 1, wherein the pharmaceutical formulation comprises a first layer and a second layer.

11. The pharmaceutical formulation of claim 10, wherein the first layer is interposed between the support frame and the second layer.

12. The pharmaceutical formulation of claim 10, wherein the first layer comprises the first active ingredient and the second active ingredient.

13. The pharmaceutical formulation of claim 10, wherein the second layer comprises the first active ingredient and the second active ingredient.

14. The pharmaceutical formulation of claim 10, wherein the first layer comprises the first active ingredient and the second active ingredient, wherein the first active ingredient and the second active ingredient are present at a first amount equal to the amount of the first active ingredient plus the amount of the second active ingredient in the first layer.

15. The pharmaceutical formulation of claim 10, wherein the second layer comprises the first active ingredient and the second active ingredient, wherein the first active ingredient and the second active ingredient are present at a second amount equal to the amount of the first active ingredient plus the amount of the second active ingredient in the second layer.

16.-26. (canceled)

27. A method of treating a disease in a subject, said method comprising the step of administering to the subject a pharmaceutical formulation comprising i) a support frame, ii) one or more bioadhesive materials, iii) a first active ingredient, and iv) a second active ingredient for treatment of the disease.

28. The method of claim 27, wherein the disease is a precancerous oral lesion.

29. The method of claim 28, wherein the precancerous oral lesion is selected from the group consisting of leukoplakia, erythroplakia, erythroleukoplakia, proliferative verrucous leukoplakia, oral lichen planus, palatal lesions in reverse smokers, dyskeratosis congenital, and any combination thereof.

30. The method of claim 27, wherein the disease is an oral potentially malignant disorder (OPMD).

31. The method of claim 27, wherein the disease is an oral cancer.

32. The method of claim 27, wherein the disease is associated with an oral dysplasia.

33. The method of claim 27, wherein the administering comprises oral administration.

34.-44. (canceled)
